SEQUENCE LISTING (469 sequences)

SEQ ID NO: 1 - DNA, 21 bp, Artificial Sequence
  GAPDH F Primer
atcaccatcttccaggagcga

SEQ ID NO: 2 - DNA, 21 bp, Artificial Sequence
  GAPDH R Primer
ttctccatggtggtgaagacg

SEQ ID NO: 3 - DNA, 20 bp, Artificial Sequence
  CDKN1A F Primer
ggaagaccatgtggacctgt

SEQ ID NO: 4 - DNA, 20 bp, Artificial Sequence
  CDKN1A R Primer
ggattagggcttcctcttgg

SEQ ID NO: 5 - DNA, 92 bp, Homo sapiens
ggctatgtggggagtattcaggagacagacaactcactcgtcaaatcctccccttcctggccaacaaagctgctgcaaccacagggatttct

SEQ ID NO: 6 - DNA, 86 bp, Homo sapiens
ggtagtctctccaattccctccttcccggaagcatgtgacaatcaacaactttgtatacttaagttcagtggacctcaatttcctc

SEQ ID NO: 7 - DNA, 35 bp, Homo sapiens
ctttgttggggtgtctaggtgctccaggtgcttct

SEQ ID NO: 8 - DNA, 51 bp, Homo sapiens
ttctgggagaggtgacctagtgagggatcagtgggaatagaggtgatattg

SEQ ID NO: 9 - DNA, 30 bp, Homo sapiens
aggtgatattgtggggcttttctggaaatt

SEQ ID NO: 10 - DNA, 38 bp, Homo sapiens
attaatgtcatcctcctgatcttttcagctgcattggg

SEQ ID NO: 11 - DNA, 40 bp, Homo sapiens
tctaacagtgctgtgtcctcctggagagtgccaactcatt

SEQ ID NO: 12 - DNA, 66 bp, Homo sapiens
gtgccaactcattctccaagtaaaaaaagccagatttgtggctcacttcgtggggaaatgtgtcca

SEQ ID NO: 13 - DNA, 21 bp, Artificial Sequence
  RAG-278
ucguggggaaauguguccatt

SEQ ID NO: 14 - DNA, 21 bp, Artificial Sequence
  RAG-278
uggacacauuuccccacgatt

SEQ ID NO: 15 - DNA, 21 bp, Artificial Sequence
  RAG-279
uucguggggaaaugugucctt

SEQ ID NO: 16 - DNA, 21 bp, Artificial Sequence
  RAG-279
ggacacauuuccccacgaatt

SEQ ID NO: 17 - DNA, 21 bp, Artificial Sequence
  RAG-280
cuucguggggaaauguguctt

SEQ ID NO: 18 - DNA, 21 bp, Artificial Sequence
  RAG-280
gacacauuuccccacgaagtt

SEQ ID NO: 19 - DNA, 21 bp, Artificial Sequence
  RAG-281
acuucguggggaaaugugutt

SEQ ID NO: 20 - DNA, 21 bp, Artificial Sequence
  RAG-281
acacauuuccccacgaagutt

SEQ ID NO: 21 - DNA, 21 bp, Artificial Sequence
  RAG-282
cacuucguggggaaaugugtt

SEQ ID NO: 22 - DNA, 21 bp, Artificial Sequence
  RAG-282
cacauuuccccacgaagugtt

SEQ ID NO: 23 - DNA, 21 bp, Artificial Sequence
  RAG-283
ucacuucguggggaaaugutt

SEQ ID NO: 24 - DNA, 21 bp, Artificial Sequence
  RAG-283
acauuuccccacgaagugatt

SEQ ID NO: 25 - DNA, 21 bp, Artificial Sequence
  RAG-284
cucacuucguggggaaaugtt

SEQ ID NO: 26 - DNA, 21 bp, Artificial Sequence
  RAG-284
cauuuccccacgaagugagtt

SEQ ID NO: 27 - DNA, 21 bp, Artificial Sequence
  RAG-285
gcucacuucguggggaaautt

SEQ ID NO: 28 - DNA, 21 bp, Artificial Sequence
  RAG-285
auuuccccacgaagugagctt

SEQ ID NO: 29 - DNA, 21 bp, Artificial Sequence
  RAG-286
ggcucacuucguggggaaatt

SEQ ID NO: 30 - DNA, 21 bp, Artificial Sequence
  RAG-286
uuuccccacgaagugagcctt

SEQ ID NO: 31 - DNA, 21 bp, Artificial Sequence
  RAG-287
uggcucacuucguggggaatt

SEQ ID NO: 32 - DNA, 21 bp, Artificial Sequence
  RAG-287
uuccccacgaagugagccatt

SEQ ID NO: 33 - DNA, 21 bp, Artificial Sequence
  RAG-288
guggcucacuucguggggatt

SEQ ID NO: 34 - DNA, 21 bp, Artificial Sequence
  RAG-288
uccccacgaagugagccactt

SEQ ID NO: 35 - DNA, 21 bp, Artificial Sequence
  RAG-289
uguggcucacuucguggggtt

SEQ ID NO: 36 - DNA, 21 bp, Artificial Sequence
  RAG-289
ccccacgaagugagccacatt

SEQ ID NO: 37 - DNA, 21 bp, Artificial Sequence
  RAG-291
uuuguggcucacuucguggtt

SEQ ID NO: 38 - DNA, 21 bp, Artificial Sequence
  RAG-291
ccacgaagugagccacaaatt

SEQ ID NO: 39 - DNA, 21 bp, Artificial Sequence
  RAG-292
auuuguggcucacuucgugtt

SEQ ID NO: 40 - DNA, 21 bp, Artificial Sequence
  RAG-292
cacgaagugagccacaaautt

SEQ ID NO: 41 - DNA, 21 bp, Artificial Sequence
  RAG-293
gauuuguggcucacuucgutt

SEQ ID NO: 42 - DNA, 21 bp, Artificial Sequence
  RAG-293
acgaagugagccacaaauctt

SEQ ID NO: 43 - DNA, 21 bp, Artificial Sequence
  RAG-294
agauuuguggcucacuucgtt

SEQ ID NO: 44 - DNA, 21 bp, Artificial Sequence
  RAG-294
cgaagugagccacaaaucutt

SEQ ID NO: 45 - DNA, 21 bp, Artificial Sequence
  RAG-295
cagauuuguggcucacuuctt

SEQ ID NO: 46 - DNA, 21 bp, Artificial Sequence
  RAG-295
gaagugagccacaaaucugtt

SEQ ID NO: 47 - DNA, 21 bp, Artificial Sequence
  RAG-296
ccagauuuguggcucacuutt

SEQ ID NO: 48 - DNA, 21 bp, Artificial Sequence
  RAG-296
aagugagccacaaaucuggtt

SEQ ID NO: 49 - DNA, 21 bp, Artificial Sequence
  RAG-297
gccagauuuguggcucacutt

SEQ ID NO: 50 - DNA, 21 bp, Artificial Sequence
  RAG-297
agugagccacaaaucuggctt

SEQ ID NO: 51 - DNA, 21 bp, Artificial Sequence
  RAG-298
agccagauuuguggcucactt

SEQ ID NO: 52 - DNA, 21 bp, Artificial Sequence
  RAG-298
gugagccacaaaucuggcutt

SEQ ID NO: 53 - DNA, 21 bp, Artificial Sequence
  RAG-299
aagccagauuuguggcucatt

SEQ ID NO: 54 - DNA, 21 bp, Artificial Sequence
  RAG-299
ugagccacaaaucuggcuutt

SEQ ID NO: 55 - DNA, 21 bp, Artificial Sequence
  RAG-300
aaagccagauuuguggcuctt

SEQ ID NO: 56 - DNA, 21 bp, Artificial Sequence
  RAG-300
gagccacaaaucuggcuuutt

SEQ ID NO: 57 - DNA, 21 bp, Artificial Sequence
  RAG-301
aaaagccagauuuguggcutt

SEQ ID NO: 58 - DNA, 21 bp, Artificial Sequence
  RAG-301
agccacaaaucuggcuuuutt

SEQ ID NO: 59 - DNA, 21 bp, Artificial Sequence
  RAG-321
caacucauucuccaaguaatt

SEQ ID NO: 60 - DNA, 21 bp, Artificial Sequence
  RAG-321
uuacuuggagaaugaguugtt

SEQ ID NO: 61 - DNA, 21 bp, Artificial Sequence
  RAG-322
ccaacucauucuccaaguatt

SEQ ID NO: 62 - DNA, 21 bp, Artificial Sequence
  RAG-322
uacuuggagaaugaguuggtt

SEQ ID NO: 63 - DNA, 21 bp, Artificial Sequence
  RAG-323
gccaacucauucuccaagutt

SEQ ID NO: 64 - DNA, 21 bp, Artificial Sequence
  RAG-323
acuuggagaaugaguuggctt

SEQ ID NO: 65 - DNA, 21 bp, Artificial Sequence
  RAG-324
ugccaacucauucuccaagtt

SEQ ID NO: 66 - DNA, 21 bp, Artificial Sequence
  RAG-324
cuuggagaaugaguuggcatt

SEQ ID NO: 67 - DNA, 21 bp, Artificial Sequence
  RAG-325
gugccaacucauucuccaatt

SEQ ID NO: 68 - DNA, 21 bp, Artificial Sequence
  RAG-325
uuggagaaugaguuggcactt

SEQ ID NO: 69 - DNA, 21 bp, Artificial Sequence
  RAG-331
uggagagugccaacucauutt

SEQ ID NO: 70 - DNA, 21 bp, Artificial Sequence
  RAG-331
aaugaguuggcacucuccatt

SEQ ID NO: 71 - DNA, 21 bp, Artificial Sequence
  RAG-332
cuggagagugccaacucautt

SEQ ID NO: 72 - DNA, 21 bp, Artificial Sequence
  RAG-332
augaguuggcacucuccagtt

SEQ ID NO: 73 - DNA, 21 bp, Artificial Sequence
  RAG-333
ccuggagagugccaacucatt

SEQ ID NO: 74 - DNA, 21 bp, Artificial Sequence
  RAG-333
ugaguuggcacucuccaggtt

SEQ ID NO: 75 - DNA, 21 bp, Artificial Sequence
  RAG-334
uccuggagagugccaacuctt

SEQ ID NO: 76 - DNA, 21 bp, Artificial Sequence
  RAG-334
gaguuggcacucuccaggatt

SEQ ID NO: 77 - DNA, 21 bp, Artificial Sequence
  RAG-335
cuccuggagagugccaacutt

SEQ ID NO: 78 - DNA, 21 bp, Artificial Sequence
  RAG-335
aguuggcacucuccaggagtt

SEQ ID NO: 79 - DNA, 21 bp, Artificial Sequence
  RAG-336
ccuccuggagagugccaactt

SEQ ID NO: 80 - DNA, 21 bp, Artificial Sequence
  RAG-336
guuggcacucuccaggaggtt

SEQ ID NO: 81 - DNA, 21 bp, Artificial Sequence
  RAG-337
uccuccuggagagugccaatt

SEQ ID NO: 82 - DNA, 21 bp, Artificial Sequence
  RAG-337
uuggcacucuccaggaggatt

SEQ ID NO: 83 - DNA, 21 bp, Artificial Sequence
  RAG-338
guccuccuggagagugccatt

SEQ ID NO: 84 - DNA, 21 bp, Artificial Sequence
  RAG-338
uggcacucuccaggaggactt

SEQ ID NO: 85 - DNA, 21 bp, Artificial Sequence
  RAG-341
uguguccuccuggagagugtt

SEQ ID NO: 86 - DNA, 21 bp, Artificial Sequence
  RAG-341
cacucuccaggaggacacatt

SEQ ID NO: 87 - DNA, 21 bp, Artificial Sequence
  RAG-342
cuguguccuccuggagagutt

SEQ ID NO: 88 - DNA, 21 bp, Artificial Sequence
  RAG-342
acucuccaggaggacacagtt

SEQ ID NO: 89 - DNA, 21 bp, Artificial Sequence
  RAG-343
gcuguguccuccuggagagtt

SEQ ID NO: 90 - DNA, 21 bp, Artificial Sequence
  RAG-343
cucuccaggaggacacagctt

SEQ ID NO: 91 - DNA, 21 bp, Artificial Sequence
  RAG-344
ugcuguguccuccuggagatt

SEQ ID NO: 92 - DNA, 21 bp, Artificial Sequence
  RAG-344
ucuccaggaggacacagcatt

SEQ ID NO: 93 - DNA, 21 bp, Artificial Sequence
  RAG-345
gugcuguguccuccuggagtt

SEQ ID NO: 94 - DNA, 21 bp, Artificial Sequence
  RAG-345
cuccaggaggacacagcactt

SEQ ID NO: 95 - DNA, 21 bp, Artificial Sequence
  RAG-346
agugcuguguccuccuggatt

SEQ ID NO: 96 - DNA, 21 bp, Artificial Sequence
  RAG-346
uccaggaggacacagcacutt

SEQ ID NO: 97 - DNA, 21 bp, Artificial Sequence
  RAG-348
acagugcuguguccuccugtt

SEQ ID NO: 98 - DNA, 21 bp, Artificial Sequence
  RAG-348
caggaggacacagcacugutt

SEQ ID NO: 99 - DNA, 21 bp, Artificial Sequence
  RAG-349
aacagugcuguguccuccutt

SEQ ID NO: 100 - DNA, 21 bp, Artificial Sequence
  RAG-349
aggaggacacagcacuguutt

SEQ ID NO: 101 - DNA, 21 bp, Artificial Sequence
  RAG-350
uaacagugcuguguccucctt

SEQ ID NO: 102 - DNA, 21 bp, Artificial Sequence
  RAG-350
ggaggacacagcacuguuatt

SEQ ID NO: 103 - DNA, 21 bp, Artificial Sequence
  RAG-351
cuaacagugcuguguccuctt

SEQ ID NO: 104 - DNA, 21 bp, Artificial Sequence
  RAG-351
gaggacacagcacuguuagtt

SEQ ID NO: 105 - DNA, 21 bp, Artificial Sequence
  RAG-352
ucuaacagugcuguguccutt

SEQ ID NO: 106 - DNA, 21 bp, Artificial Sequence
  RAG-352
aggacacagcacuguuagatt

SEQ ID NO: 107 - DNA, 21 bp, Artificial Sequence
  RAG-423
ucuuuucagcugcauugggtt

SEQ ID NO: 108 - DNA, 21 bp, Artificial Sequence
  RAG-423
cccaaugcagcugaaaagatt

SEQ ID NO: 109 - DNA, 21 bp, Artificial Sequence
  RAG-424
aucuuuucagcugcauuggtt

SEQ ID NO: 110 - DNA, 21 bp, Artificial Sequence
  RAG-424
ccaaugcagcugaaaagautt

SEQ ID NO: 111 - DNA, 21 bp, Artificial Sequence
  RAG-425
gaucuuuucagcugcauugtt

SEQ ID NO: 112 - DNA, 21 bp, Artificial Sequence
  RAG-425
caaugcagcugaaaagauctt

SEQ ID NO: 113 - DNA, 21 bp, Artificial Sequence
  RAG-426
ugaucuuuucagcugcauutt

SEQ ID NO: 114 - DNA, 21 bp, Artificial Sequence
  RAG-426
aaugcagcugaaaagaucatt

SEQ ID NO: 115 - DNA, 21 bp, Artificial Sequence
  RAG-427
cugaucuuuucagcugcautt

SEQ ID NO: 116 - DNA, 21 bp, Artificial Sequence
  RAG-427
augcagcugaaaagaucagtt

SEQ ID NO: 117 - DNA, 21 bp, Artificial Sequence
  RAG-428
ccugaucuuuucagcugcatt

SEQ ID NO: 118 - DNA, 21 bp, Artificial Sequence
  RAG-428
ugcagcugaaaagaucaggtt

SEQ ID NO: 119 - DNA, 21 bp, Artificial Sequence
  RAG-429
uccugaucuuuucagcugctt

SEQ ID NO: 120 - DNA, 21 bp, Artificial Sequence
  RAG-429
gcagcugaaaagaucaggatt

SEQ ID NO: 121 - DNA, 21 bp, Artificial Sequence
  RAG-430
cuccugaucuuuucagcugtt

SEQ ID NO: 122 - DNA, 21 bp, Artificial Sequence
  RAG-430
cagcugaaaagaucaggagtt

SEQ ID NO: 123 - DNA, 21 bp, Artificial Sequence
  RAG-431
ccuccugaucuuuucagcutt

SEQ ID NO: 124 - DNA, 21 bp, Artificial Sequence
  RAG-431
agcugaaaagaucaggaggtt

SEQ ID NO: 125 - DNA, 21 bp, Artificial Sequence
  RAG-432
uccuccugaucuuuucagctt

SEQ ID NO: 126 - DNA, 21 bp, Artificial Sequence
  RAG-432
gcugaaaagaucaggaggatt

SEQ ID NO: 127 - DNA, 21 bp, Artificial Sequence
  RAG-433
auccuccugaucuuuucagtt

SEQ ID NO: 128 - DNA, 21 bp, Artificial Sequence
  RAG-433
cugaaaagaucaggaggautt

SEQ ID NO: 129 - DNA, 21 bp, Artificial Sequence
  RAG-434
cauccuccugaucuuuucatt

SEQ ID NO: 130 - DNA, 21 bp, Artificial Sequence
  RAG-434
ugaaaagaucaggaggaugtt

SEQ ID NO: 131 - DNA, 21 bp, Artificial Sequence
  RAG-435
ucauccuccugaucuuuuctt

SEQ ID NO: 132 - DNA, 21 bp, Artificial Sequence
  RAG-435
gaaaagaucaggaggaugatt

SEQ ID NO: 133 - DNA, 21 bp, Artificial Sequence
  RAG-436
gucauccuccugaucuuuutt

SEQ ID NO: 134 - DNA, 21 bp, Artificial Sequence
  RAG-436
aaaagaucaggaggaugactt

SEQ ID NO: 135 - DNA, 21 bp, Artificial Sequence
  RAG-437
ugucauccuccugaucuuutt

SEQ ID NO: 136 - DNA, 21 bp, Artificial Sequence
  RAG-437
aaagaucaggaggaugacatt

SEQ ID NO: 137 - DNA, 21 bp, Artificial Sequence
  RAG-438
augucauccuccugaucuutt

SEQ ID NO: 138 - DNA, 21 bp, Artificial Sequence
  RAG-438
aagaucaggaggaugacautt

SEQ ID NO: 139 - DNA, 21 bp, Artificial Sequence
  RAG-439
aaugucauccuccugaucutt

SEQ ID NO: 140 - DNA, 21 bp, Artificial Sequence
  RAG-439
agaucaggaggaugacauutt

SEQ ID NO: 141 - DNA, 21 bp, Artificial Sequence
  RAG-440
uaaugucauccuccugauctt

SEQ ID NO: 142 - DNA, 21 bp, Artificial Sequence
  RAG-440
gaucaggaggaugacauuatt

SEQ ID NO: 143 - DNA, 21 bp, Artificial Sequence
  RAG-441
uuaaugucauccuccugautt

SEQ ID NO: 144 - DNA, 21 bp, Artificial Sequence
  RAG-441
aucaggaggaugacauuaatt

SEQ ID NO: 145 - DNA, 21 bp, Artificial Sequence
  RAG-442
auuaaugucauccuccugatt

SEQ ID NO: 146 - DNA, 21 bp, Artificial Sequence
  RAG-442
ucaggaggaugacauuaautt

SEQ ID NO: 147 - DNA, 21 bp, Artificial Sequence
  RAG-503
uggggcuuuucuggaaauutt

SEQ ID NO: 148 - DNA, 21 bp, Artificial Sequence
  RAG-503
aauuuccagaaaagccccatt

SEQ ID NO: 149 - DNA, 21 bp, Artificial Sequence
  RAG-504
guggggcuuuucuggaaautt

SEQ ID NO: 150 - DNA, 21 bp, Artificial Sequence
  RAG-504
auuuccagaaaagccccactt

SEQ ID NO: 151 - DNA, 21 bp, Artificial Sequence
  RAG-505
uguggggcuuuucuggaaatt

SEQ ID NO: 152 - DNA, 21 bp, Artificial Sequence
  RAG-505
uuuccagaaaagccccacatt

SEQ ID NO: 153 - DNA, 21 bp, Artificial Sequence
  RAG-506
uuguggggcuuuucuggaatt

SEQ ID NO: 154 - DNA, 21 bp, Artificial Sequence
  RAG-506
uuccagaaaagccccacaatt

SEQ ID NO: 155 - DNA, 21 bp, Artificial Sequence
  RAG-507
auuguggggcuuuucuggatt

SEQ ID NO: 156 - DNA, 21 bp, Artificial Sequence
  RAG-507
uccagaaaagccccacaautt

SEQ ID NO: 157 - DNA, 21 bp, Artificial Sequence
  RAG-508
uauuguggggcuuuucuggtt

SEQ ID NO: 158 - DNA, 21 bp, Artificial Sequence
  RAG-508
ccagaaaagccccacaauatt

SEQ ID NO: 159 - DNA, 21 bp, Artificial Sequence
  RAG-509
auauuguggggcuuuucugtt

SEQ ID NO: 160 - DNA, 21 bp, Artificial Sequence
  RAG-509
cagaaaagccccacaauautt

SEQ ID NO: 161 - DNA, 21 bp, Artificial Sequence
  RAG-510
gauauuguggggcuuuucutt

SEQ ID NO: 162 - DNA, 21 bp, Artificial Sequence
  RAG-510
agaaaagccccacaauauctt

SEQ ID NO: 163 - DNA, 21 bp, Artificial Sequence
  RAG-511
ugauauuguggggcuuuuctt

SEQ ID NO: 164 - DNA, 21 bp, Artificial Sequence
  RAG-511
gaaaagccccacaauaucatt

SEQ ID NO: 165 - DNA, 21 bp, Artificial Sequence
  RAG-512
gugauauuguggggcuuuutt

SEQ ID NO: 166 - DNA, 21 bp, Artificial Sequence
  RAG-512
aaaagccccacaauaucactt

SEQ ID NO: 167 - DNA, 21 bp, Artificial Sequence
  RAG-513
ggugauauuguggggcuuutt

SEQ ID NO: 168 - DNA, 21 bp, Artificial Sequence
  RAG-513
aaagccccacaauaucacctt

SEQ ID NO: 169 - DNA, 21 bp, Artificial Sequence
  RAG-514
aggugauauuguggggcuutt

SEQ ID NO: 170 - DNA, 21 bp, Artificial Sequence
  RAG-514
aagccccacaauaucaccutt

SEQ ID NO: 171 - DNA, 21 bp, Artificial Sequence
  RAG-522
gggaauagaggugauauugtt

SEQ ID NO: 172 - DNA, 21 bp, Artificial Sequence
  RAG-522
caauaucaccucuauuccctt

SEQ ID NO: 173 - DNA, 21 bp, Artificial Sequence
  RAG-523
ugggaauagaggugauauutt

SEQ ID NO: 174 - DNA, 21 bp, Artificial Sequence
  RAG-523
aauaucaccucuauucccatt

SEQ ID NO: 175 - DNA, 21 bp, Artificial Sequence
  RAG-524
gugggaauagaggugauautt

SEQ ID NO: 176 - DNA, 21 bp, Artificial Sequence
  RAG-524
auaucaccucuauucccactt

SEQ ID NO: 177 - DNA, 21 bp, Artificial Sequence
  RAG-525
agugggaauagaggugauatt

SEQ ID NO: 178 - DNA, 21 bp, Artificial Sequence
  RAG-525
uaucaccucuauucccacutt

SEQ ID NO: 179 - DNA, 21 bp, Artificial Sequence
  RAG-526
cagugggaauagaggugautt

SEQ ID NO: 180 - DNA, 21 bp, Artificial Sequence
  RAG-526
aucaccucuauucccacugtt

SEQ ID NO: 181 - DNA, 21 bp, Artificial Sequence
  RAG-527
ucagugggaauagaggugatt

SEQ ID NO: 182 - DNA, 21 bp, Artificial Sequence
  RAG-527
ucaccucuauucccacugatt

SEQ ID NO: 183 - DNA, 21 bp, Artificial Sequence
  RAG-528
aucagugggaauagaggugtt

SEQ ID NO: 184 - DNA, 21 bp, Artificial Sequence
  RAG-528
caccucuauucccacugautt

SEQ ID NO: 185 - DNA, 21 bp, Artificial Sequence
  RAG-529
gaucagugggaauagaggutt

SEQ ID NO: 186 - DNA, 21 bp, Artificial Sequence
  RAG-529
accucuauucccacugauctt

SEQ ID NO: 187 - DNA, 21 bp, Artificial Sequence
  RAG-530
ggaucagugggaauagaggtt

SEQ ID NO: 188 - DNA, 21 bp, Artificial Sequence
  RAG-530
ccucuauucccacugaucctt

SEQ ID NO: 189 - DNA, 21 bp, Artificial Sequence
  RAG-531
gggaucagugggaauagagtt

SEQ ID NO: 190 - DNA, 21 bp, Artificial Sequence
  RAG-531
cucuauucccacugauccctt

SEQ ID NO: 191 - DNA, 21 bp, Artificial Sequence
  RAG-532
agggaucagugggaauagatt

SEQ ID NO: 192 - DNA, 21 bp, Artificial Sequence
  RAG-532
ucuauucccacugaucccutt

SEQ ID NO: 193 - DNA, 21 bp, Artificial Sequence
  RAG-533
gagggaucagugggaauagtt

SEQ ID NO: 194 - DNA, 21 bp, Artificial Sequence
  RAG-533
cuauucccacugaucccuctt

SEQ ID NO: 195 - DNA, 21 bp, Artificial Sequence
  RAG-534
ugagggaucagugggaauatt

SEQ ID NO: 196 - DNA, 21 bp, Artificial Sequence
  RAG-534
uauucccacugaucccucatt

SEQ ID NO: 197 - DNA, 21 bp, Artificial Sequence
  RAG-535
gugagggaucagugggaautt

SEQ ID NO: 198 - DNA, 21 bp, Artificial Sequence
  RAG-535
auucccacugaucccucactt

SEQ ID NO: 199 - DNA, 21 bp, Artificial Sequence
  RAG-536
agugagggaucagugggaatt

SEQ ID NO: 200 - DNA, 21 bp, Artificial Sequence
  RAG-536
uucccacugaucccucacutt

SEQ ID NO: 201 - DNA, 21 bp, Artificial Sequence
  RAG-537
uagugagggaucagugggatt

SEQ ID NO: 202 - DNA, 21 bp, Artificial Sequence
  RAG-537
ucccacugaucccucacuatt

SEQ ID NO: 203 - DNA, 21 bp, Artificial Sequence
  RAG-538
cuagugagggaucagugggtt

SEQ ID NO: 204 - DNA, 21 bp, Artificial Sequence
  RAG-538
cccacugaucccucacuagtt

SEQ ID NO: 205 - DNA, 21 bp, Artificial Sequence
  RAG-540
accuagugagggaucagugtt

SEQ ID NO: 206 - DNA, 21 bp, Artificial Sequence
  RAG-540
cacugaucccucacuaggutt

SEQ ID NO: 207 - DNA, 21 bp, Artificial Sequence
  RAG-541
gaccuagugagggaucagutt

SEQ ID NO: 208 - DNA, 21 bp, Artificial Sequence
  RAG-541
acugaucccucacuagguctt

SEQ ID NO: 209 - DNA, 21 bp, Artificial Sequence
  RAG-542
ugaccuagugagggaucagtt

SEQ ID NO: 210 - DNA, 21 bp, Artificial Sequence
  RAG-542
cugaucccucacuaggucatt

SEQ ID NO: 211 - DNA, 21 bp, Artificial Sequence
  RAG-543
gugaccuagugagggaucatt

SEQ ID NO: 212 - DNA, 21 bp, Artificial Sequence
  RAG-543
ugaucccucacuaggucactt

SEQ ID NO: 213 - DNA, 21 bp, Artificial Sequence
  RAG-544
ggugaccuagugagggauctt

SEQ ID NO: 214 - DNA, 21 bp, Artificial Sequence
  RAG-544
gaucccucacuaggucacctt

SEQ ID NO: 215 - DNA, 21 bp, Artificial Sequence
  RAG-545
aggugaccuagugagggautt

SEQ ID NO: 216 - DNA, 21 bp, Artificial Sequence
  RAG-545
aucccucacuaggucaccutt

SEQ ID NO: 217 - DNA, 21 bp, Artificial Sequence
  RAG-546
gaggugaccuagugagggatt

SEQ ID NO: 218 - DNA, 21 bp, Artificial Sequence
  RAG-546
ucccucacuaggucaccuctt

SEQ ID NO: 219 - DNA, 21 bp, Artificial Sequence
  RAG-549
ggagaggugaccuagugagtt

SEQ ID NO: 220 - DNA, 21 bp, Artificial Sequence
  RAG-549
cucacuaggucaccucucctt

SEQ ID NO: 221 - DNA, 21 bp, Artificial Sequence
  RAG-550
gggagaggugaccuagugatt

SEQ ID NO: 222 - DNA, 21 bp, Artificial Sequence
  RAG-550
ucacuaggucaccucuccctt

SEQ ID NO: 223 - DNA, 21 bp, Artificial Sequence
  RAG-551
ugggagaggugaccuagugtt

SEQ ID NO: 224 - DNA, 21 bp, Artificial Sequence
  RAG-551
cacuaggucaccucucccatt

SEQ ID NO: 225 - DNA, 21 bp, Artificial Sequence
  RAG-552
cugggagaggugaccuagutt

SEQ ID NO: 226 - DNA, 21 bp, Artificial Sequence
  RAG-552
acuaggucaccucucccagtt

SEQ ID NO: 227 - DNA, 21 bp, Artificial Sequence
  RAG-553
ucugggagaggugaccuagtt

SEQ ID NO: 228 - DNA, 21 bp, Artificial Sequence
  RAG-553
cuaggucaccucucccagatt

SEQ ID NO: 229 - DNA, 21 bp, Artificial Sequence
  RAG-554
uucugggagaggugaccuatt

SEQ ID NO: 230 - DNA, 21 bp, Artificial Sequence
  RAG-554
uaggucaccucucccagaatt

SEQ ID NO: 231 - DNA, 21 bp, Artificial Sequence
  RAG-569
aggugcuccaggugcuucutt

SEQ ID NO: 232 - DNA, 21 bp, Artificial Sequence
  RAG-569
agaagcaccuggagcaccutt

SEQ ID NO: 233 - DNA, 21 bp, Artificial Sequence
  RAG-570
uaggugcuccaggugcuuctt

SEQ ID NO: 234 - DNA, 21 bp, Artificial Sequence
  RAG-570
gaagcaccuggagcaccuatt

SEQ ID NO: 235 - DNA, 21 bp, Artificial Sequence
  RAG-574
ugucuaggugcuccaggugtt

SEQ ID NO: 236 - DNA, 21 bp, Artificial Sequence
  RAG-574
caccuggagcaccuagacatt

SEQ ID NO: 237 - DNA, 21 bp, Artificial Sequence
  RAG-576
ggugucuaggugcuccaggtt

SEQ ID NO: 238 - DNA, 21 bp, Artificial Sequence
  RAG-576
ccuggagcaccuagacacctt

SEQ ID NO: 239 - DNA, 21 bp, Artificial Sequence
  RAG-577
gggugucuaggugcuccagtt

SEQ ID NO: 240 - DNA, 21 bp, Artificial Sequence
  RAG-577
cuggagcaccuagacaccctt

SEQ ID NO: 241 - DNA, 21 bp, Artificial Sequence
  RAG-578
ggggugucuaggugcuccatt

SEQ ID NO: 242 - DNA, 21 bp, Artificial Sequence
  RAG-578
uggagcaccuagacacccctt

SEQ ID NO: 243 - DNA, 21 bp, Artificial Sequence
  RAG-579
uggggugucuaggugcucctt

SEQ ID NO: 244 - DNA, 21 bp, Artificial Sequence
  RAG-579
ggagcaccuagacaccccatt

SEQ ID NO: 245 - DNA, 21 bp, Artificial Sequence
  RAG-580
uuggggugucuaggugcuctt

SEQ ID NO: 246 - DNA, 21 bp, Artificial Sequence
  RAG-580
gagcaccuagacaccccaatt

SEQ ID NO: 247 - DNA, 21 bp, Artificial Sequence
  RAG-583
uuguuggggugucuaggugtt

SEQ ID NO: 248 - DNA, 21 bp, Artificial Sequence
  RAG-583
caccuagacaccccaacaatt

SEQ ID NO: 249 - DNA, 21 bp, Artificial Sequence
  RAG-584
uuuguuggggugucuaggutt

SEQ ID NO: 250 - DNA, 21 bp, Artificial Sequence
  RAG-584
accuagacaccccaacaaatt

SEQ ID NO: 251 - DNA, 21 bp, Artificial Sequence
  RAG-585
cuuuguuggggugucuaggtt

SEQ ID NO: 252 - DNA, 21 bp, Artificial Sequence
  RAG-585
ccuagacaccccaacaaagtt

SEQ ID NO: 253 - DNA, 21 bp, Artificial Sequence
  RAG-650
aguggaccucaauuuccuctt

SEQ ID NO: 254 - DNA, 21 bp, Artificial Sequence
  RAG-650
gaggaaauugagguccacutt

SEQ ID NO: 255 - DNA, 21 bp, Artificial Sequence
  RAG-651
caguggaccucaauuuccutt

SEQ ID NO: 256 - DNA, 21 bp, Artificial Sequence
  RAG-651
aggaaauugagguccacugtt

SEQ ID NO: 257 - DNA, 21 bp, Artificial Sequence
  RAG-652
ucaguggaccucaauuucctt

SEQ ID NO: 258 - DNA, 21 bp, Artificial Sequence
  RAG-652
ggaaauugagguccacugatt

SEQ ID NO: 259 - DNA, 21 bp, Artificial Sequence
  RAG-653
uucaguggaccucaauuuctt

SEQ ID NO: 260 - DNA, 21 bp, Artificial Sequence
  RAG-653
gaaauugagguccacugaatt

SEQ ID NO: 261 - DNA, 21 bp, Artificial Sequence
  RAG-654
guucaguggaccucaauuutt

SEQ ID NO: 262 - DNA, 21 bp, Artificial Sequence
  RAG-654
aaauugagguccacugaactt

SEQ ID NO: 263 - DNA, 21 bp, Artificial Sequence
  RAG-655
aguucaguggaccucaauutt

SEQ ID NO: 264 - DNA, 21 bp, Artificial Sequence
  RAG-655
aauugagguccacugaacutt

SEQ ID NO: 265 - DNA, 21 bp, Artificial Sequence
  RAG-656
aaguucaguggaccucaautt

SEQ ID NO: 266 - DNA, 21 bp, Artificial Sequence
  RAG-656
auugagguccacugaacuutt

SEQ ID NO: 267 - DNA, 21 bp, Artificial Sequence
  RAG-657
uaaguucaguggaccucaatt

SEQ ID NO: 268 - DNA, 21 bp, Artificial Sequence
  RAG-657
uugagguccacugaacuuatt

SEQ ID NO: 269 - DNA, 21 bp, Artificial Sequence
  RAG-658
uuaaguucaguggaccucatt

SEQ ID NO: 270 - DNA, 21 bp, Artificial Sequence
  RAG-658
ugagguccacugaacuuaatt

SEQ ID NO: 271 - DNA, 21 bp, Artificial Sequence
  RAG-659
cuuaaguucaguggaccuctt

SEQ ID NO: 272 - DNA, 21 bp, Artificial Sequence
  RAG-659
gagguccacugaacuuaagtt

SEQ ID NO: 273 - DNA, 21 bp, Artificial Sequence
  RAG-660
acuuaaguucaguggaccutt

SEQ ID NO: 274 - DNA, 21 bp, Artificial Sequence
  RAG-660
agguccacugaacuuaagutt

SEQ ID NO: 275 - DNA, 21 bp, Artificial Sequence
  RAG-661
uacuuaaguucaguggacctt

SEQ ID NO: 276 - DNA, 21 bp, Artificial Sequence
  RAG-661
gguccacugaacuuaaguatt

SEQ ID NO: 277 - DNA, 21 bp, Artificial Sequence
  RAG-662
auacuuaaguucaguggactt

SEQ ID NO: 278 - DNA, 21 bp, Artificial Sequence
  RAG-662
guccacugaacuuaaguautt

SEQ ID NO: 279 - DNA, 21 bp, Artificial Sequence
  RAG-682
gugacaaucaacaacuuugtt

SEQ ID NO: 280 - DNA, 21 bp, Artificial Sequence
  RAG-682
caaaguuguugauugucactt

SEQ ID NO: 281 - DNA, 21 bp, Artificial Sequence
  RAG-685
caugugacaaucaacaacutt

SEQ ID NO: 282 - DNA, 21 bp, Artificial Sequence
  RAG-685
aguuguugauugucacaugtt

SEQ ID NO: 283 - DNA, 21 bp, Artificial Sequence
  RAG-686
gcaugugacaaucaacaactt

SEQ ID NO: 284 - DNA, 21 bp, Artificial Sequence
  RAG-686
guuguugauugucacaugctt

SEQ ID NO: 285 - DNA, 21 bp, Artificial Sequence
  RAG-687
agcaugugacaaucaacaatt

SEQ ID NO: 286 - DNA, 21 bp, Artificial Sequence
  RAG-687
uuguugauugucacaugcutt

SEQ ID NO: 287 - DNA, 21 bp, Artificial Sequence
  RAG-688
aagcaugugacaaucaacatt

SEQ ID NO: 288 - DNA, 21 bp, Artificial Sequence
  RAG-688
uguugauugucacaugcuutt

SEQ ID NO: 289 - DNA, 21 bp, Artificial Sequence
  RAG-689
gaagcaugugacaaucaactt

SEQ ID NO: 290 - DNA, 21 bp, Artificial Sequence
  RAG-689
guugauugucacaugcuuctt

SEQ ID NO: 291 - DNA, 21 bp, Artificial Sequence
  RAG-690
ggaagcaugugacaaucaatt

SEQ ID NO: 292 - DNA, 21 bp, Artificial Sequence
  RAG-690
uugauugucacaugcuucctt

SEQ ID NO: 293 - DNA, 21 bp, Artificial Sequence
  RAG-691
cggaagcaugugacaaucatt

SEQ ID NO: 294 - DNA, 21 bp, Artificial Sequence
  RAG-691
ugauugucacaugcuuccgtt

SEQ ID NO: 295 - DNA, 21 bp, Artificial Sequence
  RAG-692
ccggaagcaugugacaauctt

SEQ ID NO: 296 - DNA, 21 bp, Artificial Sequence
  RAG-692
gauugucacaugcuuccggtt

SEQ ID NO: 297 - DNA, 21 bp, Artificial Sequence
  RAG-693
cccggaagcaugugacaautt

SEQ ID NO: 298 - DNA, 21 bp, Artificial Sequence
  RAG-693
auugucacaugcuuccgggtt

SEQ ID NO: 299 - DNA, 21 bp, Artificial Sequence
  RAG-694
ucccggaagcaugugacaatt

SEQ ID NO: 300 - DNA, 21 bp, Artificial Sequence
  RAG-694
uugucacaugcuuccgggatt

SEQ ID NO: 301 - DNA, 21 bp, Artificial Sequence
  RAG-695
uucccggaagcaugugacatt

SEQ ID NO: 302 - DNA, 21 bp, Artificial Sequence
  RAG-695
ugucacaugcuuccgggaatt

SEQ ID NO: 303 - DNA, 21 bp, Artificial Sequence
  RAG-696
cuucccggaagcaugugactt

SEQ ID NO: 304 - DNA, 21 bp, Artificial Sequence
  RAG-696
gucacaugcuuccgggaagtt

SEQ ID NO: 305 - DNA, 21 bp, Artificial Sequence
  RAG-697
ccuucccggaagcaugugatt

SEQ ID NO: 306 - DNA, 21 bp, Artificial Sequence
  RAG-697
ucacaugcuuccgggaaggtt

SEQ ID NO: 307 - DNA, 21 bp, Artificial Sequence
  RAG-698
uccuucccggaagcaugugtt

SEQ ID NO: 308 - DNA, 21 bp, Artificial Sequence
  RAG-698
cacaugcuuccgggaaggatt

SEQ ID NO: 309 - DNA, 21 bp, Artificial Sequence
  RAG-699
cuccuucccggaagcaugutt

SEQ ID NO: 310 - DNA, 21 bp, Artificial Sequence
  RAG-699
acaugcuuccgggaaggagtt

SEQ ID NO: 311 - DNA, 21 bp, Artificial Sequence
  RAG-700
ccuccuucccggaagcaugtt

SEQ ID NO: 312 - DNA, 21 bp, Artificial Sequence
  RAG-700
caugcuuccgggaaggaggtt

SEQ ID NO: 313 - DNA, 21 bp, Artificial Sequence
  RAG-701
cccuccuucccggaagcautt

SEQ ID NO: 314 - DNA, 21 bp, Artificial Sequence
  RAG-701
augcuuccgggaaggagggtt

SEQ ID NO: 315 - DNA, 21 bp, Artificial Sequence
  RAG-702
ucccuccuucccggaagcatt

SEQ ID NO: 316 - DNA, 21 bp, Artificial Sequence
  RAG-702
ugcuuccgggaaggagggatt

SEQ ID NO: 317 - DNA, 21 bp, Artificial Sequence
  RAG-704
auucccuccuucccggaagtt

SEQ ID NO: 318 - DNA, 21 bp, Artificial Sequence
  RAG-704
cuuccgggaaggagggaautt

SEQ ID NO: 319 - DNA, 21 bp, Artificial Sequence
  RAG-705
aauucccuccuucccggaatt

SEQ ID NO: 320 - DNA, 21 bp, Artificial Sequence
  RAG-705
uuccgggaaggagggaauutt

SEQ ID NO: 321 - DNA, 21 bp, Artificial Sequence
  RAG-710
ucuccaauucccuccuucctt

SEQ ID NO: 322 - DNA, 21 bp, Artificial Sequence
  RAG-710
ggaaggagggaauuggagatt

SEQ ID NO: 323 - DNA, 21 bp, Artificial Sequence
  RAG-711
cucuccaauucccuccuuctt

SEQ ID NO: 324 - DNA, 21 bp, Artificial Sequence
  RAG-711
gaaggagggaauuggagagtt

SEQ ID NO: 325 - DNA, 21 bp, Artificial Sequence
  RAG-712
ucucuccaauucccuccuutt

SEQ ID NO: 326 - DNA, 21 bp, Artificial Sequence
  RAG-712
aaggagggaauuggagagatt

SEQ ID NO: 327 - DNA, 21 bp, Artificial Sequence
  RAG-713
gucucuccaauucccuccutt

SEQ ID NO: 328 - DNA, 21 bp, Artificial Sequence
  RAG-713
aggagggaauuggagagactt

SEQ ID NO: 329 - DNA, 21 bp, Artificial Sequence
  RAG-714
agucucuccaauucccucctt

SEQ ID NO: 330 - DNA, 21 bp, Artificial Sequence
  RAG-714
ggagggaauuggagagacutt

SEQ ID NO: 331 - DNA, 21 bp, Artificial Sequence
  RAG-715
uagucucuccaauucccuctt

SEQ ID NO: 332 - DNA, 21 bp, Artificial Sequence
  RAG-715
gagggaauuggagagacuatt

SEQ ID NO: 333 - DNA, 21 bp, Artificial Sequence
  RAG-716
guagucucuccaauucccutt

SEQ ID NO: 334 - DNA, 21 bp, Artificial Sequence
  RAG-716
agggaauuggagagacuactt

SEQ ID NO: 335 - DNA, 21 bp, Artificial Sequence
  RAG-717
gguagucucuccaauuccctt

SEQ ID NO: 336 - DNA, 21 bp, Artificial Sequence
  RAG-717
gggaauuggagagacuacctt

SEQ ID NO: 337 - DNA, 21 bp, Artificial Sequence
  RAG-820
ugcaaccacagggauuucutt

SEQ ID NO: 338 - DNA, 21 bp, Artificial Sequence
  RAG-820
agaaaucccugugguugcatt

SEQ ID NO: 339 - DNA, 21 bp, Artificial Sequence
  RAG-821
cugcaaccacagggauuuctt

SEQ ID NO: 340 - DNA, 21 bp, Artificial Sequence
  RAG-821
gaaaucccugugguugcagtt

SEQ ID NO: 341 - DNA, 21 bp, Artificial Sequence
  RAG-822
gcugcaaccacagggauuutt

SEQ ID NO: 342 - DNA, 21 bp, Artificial Sequence
  RAG-822
aaaucccugugguugcagctt

SEQ ID NO: 343 - DNA, 21 bp, Artificial Sequence
  RAG-823
ugcugcaaccacagggauutt

SEQ ID NO: 344 - DNA, 21 bp, Artificial Sequence
  RAG-823
aaucccugugguugcagcatt

SEQ ID NO: 345 - DNA, 21 bp, Artificial Sequence
  RAG-824
cugcugcaaccacagggautt

SEQ ID NO: 346 - DNA, 21 bp, Artificial Sequence
  RAG-824
aucccugugguugcagcagtt

SEQ ID NO: 347 - DNA, 21 bp, Artificial Sequence
  RAG-825
gcugcugcaaccacagggatt

SEQ ID NO: 348 - DNA, 21 bp, Artificial Sequence
  RAG-825
ucccugugguugcagcagctt

SEQ ID NO: 349 - DNA, 21 bp, Artificial Sequence
  RAG-826
agcugcugcaaccacagggtt

SEQ ID NO: 350 - DNA, 21 bp, Artificial Sequence
  RAG-826
cccugugguugcagcagcutt

SEQ ID NO: 351 - DNA, 21 bp, Artificial Sequence
  RAG-828
aaagcugcugcaaccacagtt

SEQ ID NO: 352 - DNA, 21 bp, Artificial Sequence
  RAG-828
cugugguugcagcagcuuutt

SEQ ID NO: 353 - DNA, 21 bp, Artificial Sequence
  RAG-829
caaagcugcugcaaccacatt

SEQ ID NO: 354 - DNA, 21 bp, Artificial Sequence
  RAG-829
ugugguugcagcagcuuugtt

SEQ ID NO: 355 - DNA, 21 bp, Artificial Sequence
  RAG-830
acaaagcugcugcaaccactt

SEQ ID NO: 356 - DNA, 21 bp, Artificial Sequence
  RAG-830
gugguugcagcagcuuugutt

SEQ ID NO: 357 - DNA, 21 bp, Artificial Sequence
  RAG-831
aacaaagcugcugcaaccatt

SEQ ID NO: 358 - DNA, 21 bp, Artificial Sequence
  RAG-831
ugguugcagcagcuuuguutt

SEQ ID NO: 359 - DNA, 21 bp, Artificial Sequence
  RAG-832
caacaaagcugcugcaacctt

SEQ ID NO: 360 - DNA, 21 bp, Artificial Sequence
  RAG-832
gguugcagcagcuuuguugtt

SEQ ID NO: 361 - DNA, 21 bp, Artificial Sequence
  RAG-833
ccaacaaagcugcugcaactt

SEQ ID NO: 362 - DNA, 21 bp, Artificial Sequence
  RAG-833
guugcagcagcuuuguuggtt

SEQ ID NO: 363 - DNA, 21 bp, Artificial Sequence
  RAG-834
gccaacaaagcugcugcaatt

SEQ ID NO: 364 - DNA, 21 bp, Artificial Sequence
  RAG-834
uugcagcagcuuuguuggctt

SEQ ID NO: 365 - DNA, 21 bp, Artificial Sequence
  RAG-835
ggccaacaaagcugcugcatt

SEQ ID NO: 366 - DNA, 21 bp, Artificial Sequence
  RAG-835
ugcagcagcuuuguuggcctt

SEQ ID NO: 367 - DNA, 21 bp, Artificial Sequence
  RAG-836
uggccaacaaagcugcugctt

SEQ ID NO: 368 - DNA, 21 bp, Artificial Sequence
  RAG-836
gcagcagcuuuguuggccatt

SEQ ID NO: 369 - DNA, 21 bp, Artificial Sequence
  RAG-837
cuggccaacaaagcugcugtt

SEQ ID NO: 370 - DNA, 21 bp, Artificial Sequence
  RAG-837
cagcagcuuuguuggccagtt

SEQ ID NO: 371 - DNA, 21 bp, Artificial Sequence
  RAG-838
ccuggccaacaaagcugcutt

SEQ ID NO: 372 - DNA, 21 bp, Artificial Sequence
  RAG-838
agcagcuuuguuggccaggtt

SEQ ID NO: 373 - DNA, 21 bp, Artificial Sequence
  RAG-840
uuccuggccaacaaagcugtt

SEQ ID NO: 374 - DNA, 21 bp, Artificial Sequence
  RAG-840
cagcuuuguuggccaggaatt

SEQ ID NO: 375 - DNA, 21 bp, Artificial Sequence
  RAG-841
cuuccuggccaacaaagcutt

SEQ ID NO: 376 - DNA, 21 bp, Artificial Sequence
  RAG-841
agcuuuguuggccaggaagtt

SEQ ID NO: 377 - DNA, 21 bp, Artificial Sequence
  RAG-843
cccuuccuggccaacaaagtt

SEQ ID NO: 378 - DNA, 21 bp, Artificial Sequence
  RAG-843
cuuuguuggccaggaagggtt

SEQ ID NO: 379 - DNA, 21 bp, Artificial Sequence
  RAG-844
ccccuuccuggccaacaaatt

SEQ ID NO: 380 - DNA, 21 bp, Artificial Sequence
  RAG-844
uuuguuggccaggaaggggtt

SEQ ID NO: 381 - DNA, 21 bp, Artificial Sequence
  RAG-845
uccccuuccuggccaacaatt

SEQ ID NO: 382 - DNA, 21 bp, Artificial Sequence
  RAG-845
uuguuggccaggaaggggatt

SEQ ID NO: 383 - DNA, 21 bp, Artificial Sequence
  RAG-846
cuccccuuccuggccaacatt

SEQ ID NO: 384 - DNA, 21 bp, Artificial Sequence
  RAG-846
uguuggccaggaaggggagtt

SEQ ID NO: 385 - DNA, 21 bp, Artificial Sequence
  RAG-848
uccuccccuuccuggccaatt

SEQ ID NO: 386 - DNA, 21 bp, Artificial Sequence
  RAG-848
uuggccaggaaggggaggatt

SEQ ID NO: 387 - DNA, 21 bp, Artificial Sequence
  RAG-849
auccuccccuuccuggccatt

SEQ ID NO: 388 - DNA, 21 bp, Artificial Sequence
  RAG-849
uggccaggaaggggaggautt

SEQ ID NO: 389 - DNA, 21 bp, Artificial Sequence
  RAG-853
ucaaauccuccccuuccugtt

SEQ ID NO: 390 - DNA, 21 bp, Artificial Sequence
  RAG-853
caggaaggggaggauuugatt

SEQ ID NO: 391 - DNA, 21 bp, Artificial Sequence
  RAG-854
gucaaauccuccccuuccutt

SEQ ID NO: 392 - DNA, 21 bp, Artificial Sequence
  RAG-854
aggaaggggaggauuugactt

SEQ ID NO: 393 - DNA, 21 bp, Artificial Sequence
  RAG-855
cgucaaauccuccccuucctt

SEQ ID NO: 394 - DNA, 21 bp, Artificial Sequence
  RAG-855
ggaaggggaggauuugacgtt

SEQ ID NO: 395 - DNA, 21 bp, Artificial Sequence
  RAG-856
ucgucaaauccuccccuuctt

SEQ ID NO: 396 - DNA, 21 bp, Artificial Sequence
  RAG-856
gaaggggaggauuugacgatt

SEQ ID NO: 397 - DNA, 21 bp, Artificial Sequence
  RAG-857
cucgucaaauccuccccuutt

SEQ ID NO: 398 - DNA, 21 bp, Artificial Sequence
  RAG-857
aaggggaggauuugacgagtt

SEQ ID NO: 399 - DNA, 21 bp, Artificial Sequence
  RAG-858
acucgucaaauccuccccutt

SEQ ID NO: 400 - DNA, 21 bp, Artificial Sequence
  RAG-858
aggggaggauuugacgagutt

SEQ ID NO: 401 - DNA, 21 bp, Artificial Sequence
  RAG-860
ucacucgucaaauccuccctt

SEQ ID NO: 402 - DNA, 21 bp, Artificial Sequence
  RAG-860
gggaggauuugacgagugatt

SEQ ID NO: 403 - DNA, 21 bp, Artificial Sequence
  RAG-861
cucacucgucaaauccucctt

SEQ ID NO: 404 - DNA, 21 bp, Artificial Sequence
  RAG-861
ggaggauuugacgagugagtt

SEQ ID NO: 405 - DNA, 21 bp, Artificial Sequence
  RAG-862
acucacucgucaaauccuctt

SEQ ID NO: 406 - DNA, 21 bp, Artificial Sequence
  RAG-862
gaggauuugacgagugagutt

SEQ ID NO: 407 - DNA, 21 bp, Artificial Sequence
  RAG-864
caacucacucgucaaaucctt

SEQ ID NO: 408 - DNA, 21 bp, Artificial Sequence
  RAG-864
ggauuugacgagugaguugtt

SEQ ID NO: 409 - DNA, 21 bp, Artificial Sequence
  RAG-865
acaacucacucgucaaauctt

SEQ ID NO: 410 - DNA, 21 bp, Artificial Sequence
  RAG-865
gauuugacgagugaguugutt

SEQ ID NO: 411 - DNA, 21 bp, Artificial Sequence
  RAG-866
gacaacucacucgucaaautt

SEQ ID NO: 412 - DNA, 21 bp, Artificial Sequence
  RAG-866
auuugacgagugaguuguctt

SEQ ID NO: 413 - DNA, 21 bp, Artificial Sequence
  RAG-867
agacaacucacucgucaaatt

SEQ ID NO: 414 - DNA, 21 bp, Artificial Sequence
  RAG-867
uuugacgagugaguugucutt

SEQ ID NO: 415 - DNA, 21 bp, Artificial Sequence
  RAG-868
cagacaacucacucgucaatt

SEQ ID NO: 416 - DNA, 21 bp, Artificial Sequence
  RAG-868
uugacgagugaguugucugtt

SEQ ID NO: 417 - DNA, 21 bp, Artificial Sequence
  RAG-869
acagacaacucacucgucatt

SEQ ID NO: 418 - DNA, 21 bp, Artificial Sequence
  RAG-869
ugacgagugaguugucugutt

SEQ ID NO: 419 - DNA, 21 bp, Artificial Sequence
  RAG-870
gacagacaacucacucguctt

SEQ ID NO: 420 - DNA, 21 bp, Artificial Sequence
  RAG-870
gacgagugaguugucuguctt

SEQ ID NO: 421 - DNA, 21 bp, Artificial Sequence
  RAG-871
agacagacaacucacucgutt

SEQ ID NO: 422 - DNA, 21 bp, Artificial Sequence
  RAG-871
acgagugaguugucugucutt

SEQ ID NO: 423 - DNA, 21 bp, Artificial Sequence
  RAG-872
gagacagacaacucacucgtt

SEQ ID NO: 424 - DNA, 21 bp, Artificial Sequence
  RAG-872
cgagugaguugucugucuctt

SEQ ID NO: 425 - DNA, 21 bp, Artificial Sequence
  RAG-873
ggagacagacaacucacuctt

SEQ ID NO: 426 - DNA, 21 bp, Artificial Sequence
  RAG-873
gagugaguugucugucucctt

SEQ ID NO: 427 - DNA, 21 bp, Artificial Sequence
  RAG-874
aggagacagacaacucacutt

SEQ ID NO: 428 - DNA, 21 bp, Artificial Sequence
  RAG-874
agugaguugucugucuccutt

SEQ ID NO: 429 - DNA, 21 bp, Artificial Sequence
  RAG-875
caggagacagacaacucactt

SEQ ID NO: 430 - DNA, 21 bp, Artificial Sequence
  RAG-875
gugaguugucugucuccugtt

SEQ ID NO: 431 - DNA, 21 bp, Artificial Sequence
  RAG-876
ucaggagacagacaacucatt

SEQ ID NO: 432 - DNA, 21 bp, Artificial Sequence
  RAG-876
ugaguugucugucuccugatt

SEQ ID NO: 433 - DNA, 21 bp, Artificial Sequence
  RAG-877
uucaggagacagacaacuctt

SEQ ID NO: 434 - DNA, 20 bp, Artificial Sequence
  RAG-877
gaguugucugucuccugaat

SEQ ID NO: 435 - DNA, 21 bp, Artificial Sequence
  RAG-878
auucaggagacagacaacutt

SEQ ID NO: 436 - DNA, 21 bp, Artificial Sequence
  RAG-878
aguugucugucuccugaautt

SEQ ID NO: 437 - DNA, 21 bp, Artificial Sequence
  RAG-879
uauucaggagacagacaactt

SEQ ID NO: 438 - DNA, 21 bp, Artificial Sequence
  RAG-879
guugucugucuccugaauatt

SEQ ID NO: 439 - DNA, 21 bp, Artificial Sequence
  RAG-880
guauucaggagacagacaatt

SEQ ID NO: 440 - DNA, 21 bp, Artificial Sequence
  RAG-880
uugucugucuccugaauactt

SEQ ID NO: 441 - DNA, 21 bp, Artificial Sequence
  RAG-881
aguauucaggagacagacatt

SEQ ID NO: 442 - DNA, 21 bp, Artificial Sequence
  RAG-881
ugucugucuccugaauacutt

SEQ ID NO: 443 - DNA, 21 bp, Artificial Sequence
  RAG-882
gaguauucaggagacagactt

SEQ ID NO: 444 - DNA, 21 bp, Artificial Sequence
  RAG-882
gucugucuccugaauacuctt

SEQ ID NO: 445 - DNA, 21 bp, Artificial Sequence
  RAG-883
ggaguauucaggagacagatt

SEQ ID NO: 446 - DNA, 21 bp, Artificial Sequence
  RAG-883
ucugucuccugaauacucctt

SEQ ID NO: 447 - DNA, 21 bp, Artificial Sequence
  RAG-884
gggaguauucaggagacagtt

SEQ ID NO: 448 - DNA, 21 bp, Artificial Sequence
  RAG-884
cugucuccugaauacuccctt

SEQ ID NO: 449 - DNA, 21 bp, Artificial Sequence
  RAG-885
ggggaguauucaggagacatt

SEQ ID NO: 450 - DNA, 21 bp, Artificial Sequence
  RAG-885
ugucuccugaauacucccctt

SEQ ID NO: 451 - DNA, 21 bp, Artificial Sequence
  RAG-886
uggggaguauucaggagactt

SEQ ID NO: 452 - DNA, 21 bp, Artificial Sequence
  RAG-886
gucuccugaauacuccccatt

SEQ ID NO: 453 - DNA, 21 bp, Artificial Sequence
  RAG-887
guggggaguauucaggagatt

SEQ ID NO: 454 - DNA, 21 bp, Artificial Sequence
  RAG-887
ucuccugaauacuccccactt

SEQ ID NO: 455 - DNA, 21 bp, Artificial Sequence
  RAG-888
uguggggaguauucaggagtt

SEQ ID NO: 456 - DNA, 21 bp, Artificial Sequence
  RAG-888
cuccugaauacuccccacatt

SEQ ID NO: 457 - DNA, 21 bp, Artificial Sequence
  RAG-889
auguggggaguauucaggatt

SEQ ID NO: 458 - DNA, 21 bp, Artificial Sequence
  RAG-889
uccugaauacuccccacautt

SEQ ID NO: 459 - DNA, 21 bp, Artificial Sequence
  RAG-890
uauguggggaguauucaggtt

SEQ ID NO: 460 - DNA, 21 bp, Artificial Sequence
  RAG-890
ccugaauacuccccacauatt

SEQ ID NO: 461 - DNA, 21 bp, Artificial Sequence
  RAG-891
cuauguggggaguauucagtt

SEQ ID NO: 462 - DNA, 21 bp, Artificial Sequence
  RAG-891
cugaauacuccccacauagtt

SEQ ID NO: 463 - DNA, 21 bp, Artificial Sequence
  RAG-892
gcuauguggggaguauucatt

SEQ ID NO: 464 - DNA, 21 bp, Artificial Sequence
  RAG-892
ugaauacuccccacauagctt

SEQ ID NO: 465 - DNA, 21 bp, Artificial Sequence
  RAG-893
ggcuauguggggaguauuctt

SEQ ID NO: 466 - DNA, 21 bp, Artificial Sequence
  RAG-893
gaauacuccccacauagcctt

SEQ ID NO: 467 - DNA, 21 bp, Artificial Sequence
  RAG-894
gggcuauguggggaguauutt

SEQ ID NO: 468 - DNA, 21 bp, Artificial Sequence
  RAG-894
aauacuccccacauagccctt

SEQ ID NO: 469 - DNA, 1003 bp, Artificial Sequence
  p21 promoter
gcaggaggcaaaagtcctgtgttccaactatagtcatttctttgctgcatgatctgagttaggtcaccagacttctctgagccccagtttccccagcagtgtatacgggctatgtggggagtattcaggagacagacaactcactcgtcaaatcctccccttcctggccaacaaagctgctgcaaccacagggatttcttctgttcaggtgagtgtagggtgtagggagattggttcaatgtccaattcttctgtttccctggagatcaggttgcccttttttggtagtctctccaattccctccttcccggaagcatgtgacaatcaacaactttgtatacttaagttcagtggacctcaatttcctcatctgtgaaataaacgggactgaaaaatcattctggcctcaagatgctttgttggggtgtctaggtgctccaggtgcttctgggagaggtgacctagtgagggatcagtgggaatagaggtgatattgtggggcttttctggaaattgcagagaggtgcatcgtttttataatttatgaatttttatgtattaatgtcatcctcctgatcttttcagctgcattgggtaaatccttgcctgccagagtgggtcagcggtgagccagaaagggggctcattctaacagtgctgtgtcctcctggagagtgccaactcattctccaagtaaaaaaagccagatttgtggctcacttcgtggggaaatgtgtccagcgcaccaacgcaggcgagggactgggggaggagggaagtgccctcctgcagcacgcgaggttccgggaccggctggcctgctggaactcggccaggctcagctggctcggcgctgggcagccaggagcctgggccccggggagggcggtcccgggcggcgcggtgggccgagcgcgggtcccgcctccttgaggcgggcccgggcggggcggttgtatatcagggccgcgctgagctgcgccagctgaggtgtgagcagct

(19) United States
(12) Patent Application Publication
(10) Pub. No.: US 2021/0024915 A1
(43) Pub. Date: Jan. 28, 2021

(54) METHOD FOR ACTIVATING P21 GENE EXPRESSION

(71) Applicant: RACTIGEN THERAPEUTICS, Nantong City, Jiangsu Province (CN)

(72) Inventors: Longcheng Li, Nantong City (CN); Moorim Kang, Nantong Ciry (CN); Jiancheng Wu, Nantong City (CN)

(21) Appl. No.: 15/733,357

(22) PCT Filed: Apr. 10, 2019
(86) PCT No.: PCT/CN2019/082126

§ 371 Date: Jul. 9, 2020

(30) Foreign Application Priority Data
Apr. 10, 2018 (CN) 201810316672.1

(51) Int. Cl. C12N 15/10; A61K 47/69; A61K 31/713; C12N 15/87

(52) U.S. Cl. CPC C12N 15/102; A61K 47/6911; A61K 31/713; C12N 2310/33; C12N 2310/321; C12N 2310/31; C12N 15/87

(57) ABSTRACT

The present invention provides a saRNA for activating or upregulating human p21 expression, the saRNA comprises a sense oligonucleotide strand and an antisense oligonucleotide strand, and the sense nucleic acid strand or the antisense nucleic acid strand has more than 75% homology with any continuous fragment of 16 to 35 nucleotides in length in the target gene sequence of a human p21 promoter, wherein the target nucleic acid sequence of the human p21 promoter is selected from a group consisting of SEQ ID NOs: 5, 6, 7, 8, 9, 10, 11, and 12.

### FIELD OF THE INVENTION

The present invention relates to the field of molecular biology, and in particular to up-regulating gene expression with a double-stranded small RNA targeting a gene promoter.

### BACKGROUND OF THE INVENTION

Double-stranded small nucleic acid molecules including chemically-synthesized oligoribonucleotides (such as small activating RNA (saRNA)) and naturally occurring oligoribonucleotides (such as micro ribonucleic acid (miRNA)) have been proven to be capable of targeting regulatory sequences (such as promoter sequences) of protein-coding genes in a sequence-specific manner to up-regulate gene expression at the transcriptional and epigenetic level, a phenomenon known as RNA activation (RNAa) (Li, Okino et al. (2006) Proc Natl Acad Sci USA 103:17337-17342; Janowski, Younger et al. (2007) Nat Chem Biol 3:166-173; Place, Li et al. (2008) Proc Natl Acad Sci USA 105:1608-1613; Huang, Place et al. (2012) Nucleic Acids Res 40:1695-1707; Li (2017) Adv Exp Med Biol 983:1-20). Studies have shown that RNA-a is an endogenous molecular mechanism evolutionarily conserved from Caenorhabditis elegans to the human being (Huang, Qin et al. (2010) PLoS One 5:e8848; Seth, Shirayama et al. (2013) Dev Cell 27:656-663; Turner, Jiao et al. (2014) Cell Cycle 13:772-781).

Safe strategies to selectively enhance gene and/or protein production remain a challenge in gene therapy. Viral-based systems have inherent drawbacks including adverse effects on host genome integrity and immunological consequences. RNAa, with the advantages of being able to activate endogenous genes without the risk of altering the genome, represents a new strategy to stimulate target gene expression.

Cyclin-dependent kinase (CDK) inhibitor p21^{WAF1/CIP1} (p21) is a mediator of several anti-growth pathways and considered a potent tumor suppressor gene in cancer cells (Harper, Adami et al. (1993) Cell 75:805-816). In fact, overexpression of p21 by ectopic vectors or stimulation of endogenous transcription inhibits tumor growth both in vitro and in vivo (Harper, Adami et al. (1993) Cell 75:805-816; Eastham, Hall et al. (1995) Cancer Res 55:5151-5155; Wu, Bellas et al. (1998) J Exp Med 187:1671-1679; Harrington, Spitzweg et al. (2001) J Urol 166:1220-1233). As such, selective activation of p21 can possess therapeutic application for regulating cell growth and treatment of disease (e.g. cancer).

### SUMMARY OF THE INVENTION

The present invention provides a saRNA, wherein one strand of the saRNA has at least 75% homology or complementarity with any continuous fragment of 16 to 35 nucleotides in length in a nucleic acid sequence selected from the group consisting of SEQ ID NOs: 5, 6, 7, 8, 9, 10, 11, and 12, and wherein the saRNA activates or upregulates the expression of p21 by targeting the sequence of a human p21 promoter.

In certain embodiments, a target gene sequence of the human p21 is selected from a group consisting of SEQ ID NO: 5-12, wherein the sequence of the human p21 promoter is from −893 bp to −801 bp (SEQ ID NO: 5), −717 bp to −632 bp (SEQ ID NO: 6), −585 bp to −551 bp (SEQ ID NO: 7), −554 bp to −504 bp (SEQ ID NO: 8), −514 bp to −485 bp (SEQ ID NO: 9), −442 bp to −405 bp (SEQ ID NO: 10), −352 bp to −313 bp (SEQ ID NO: 11), or −325 bp to −260 bp (SEQ ID NO: 12) upstream of the transcription start site (TSS) respectively.

In certain embodiments, the saRNA comprises a sense nucleic acid strand and an antisense nucleic acid strand. The sense nucleic acid strand and the antisense nucleic acid strand can contain complementary regions capable of forming a double-stranded nucleic acid structure, and the sense nucleic acid strand or the antisense nucleic acid strand has more than 75%, more than 80%, more than 90%, more than 95%, more than 99%, or 100% homology with any continuous fragment of 16 to 35 nucleotides in length in a sequence of a human p21 promoter.

In certain embodiments, the sense nucleic acid strand and the antisense nucleic acid strand are on two different nucleic acid strands; or the sense nucleic acid strand and the antisense nucleic acid strand are on the same nucleic acid strand, forming a hairpin single-stranded nucleic acid molecule, wherein the complementary regions of the sense nucleic acid strand and the antisense nucleic acid strand form a double-stranded nucleic acid structure.

In certain embodiments, at least one strand of the saRNA has a 3′ overhang of 0 to 6 nucleotides in length; or both strands of the saRNA have a 3′ overhang of 2 to 3 nucleotides in length.

In certain embodiments, the sense nucleic acid strand or the antisense nucleic acid strand has 16 to 35 nucleotides, such as 16, 17, 18, 19, 20, 21, 22, 23, 24, 25, 26, 27, 28, 29, 30, 31, 32, 33, 34, or 35 nucleotides, in length.

In certain embodiments, the sense nucleic acid strand or the antisense nucleic acid strand has at least 75%, such as 80%, 85%, 90%, 95%, 99%, or 100%, homology with a nucleic acid sequence selected from the group consisting of SEQ ID NOs: 13-30, 35-46, 59-62, 67-74, 77-80, 85-96, 103-108, 111-118, 121-132, 139-140, 147-180, 185-186, 189-190, 195-198, 201-202, 209-212, 215-218, 225-240, 243-246, 249-258, 261-262, 265-270, 275-280, 283-300, 303-308, 317-320, 323-324, 329-348, 351-352, 357-358, 361-366, 371-392, 399-400, 405-412, 415-416, 419-424, 429-432, 439-442, 447-450, 453-458, and 463-468.

In certain embodiments, the nucleotide sequence of the sense nucleic acid strand or the antisense nucleic acid strand is selected from the group consisting of SEQ ID NOs: 13-30, 35-46, 59-62, 67-74, 77-80, 85-96, 103-108, 111-118, 121-132, 139-140, 147-180, 185-186, 189-190, 195-198, 201-202, 209-212, 215-218, 225-240, 243-246, 249-258, 261-262, 265-270, 275-280, 283-300, 303-308, 317-320, 323-324, 329-348, 351-352, 357-358, 361-366, 371-392, 399-400, 405-412, 415-416, 419-424, 429-432, 439-442, 447-450, 453-458, and 463-468.

The present invention further provides a method for preparing the saRNA as described by any one of the aforementioned examples, wherein the method comprises the following steps: 1) selecting a sequence containing 19 bases as a target site by using the sequence of a promoter of a target gene as a template; 2) synthesizing a RNA sequence having more than 75% homology with the sequence of the target site obtained in step 1) to obtain a sense oligonucleotide strand; 3) synthesizing a sequence complementary with the sense oligonucleotide strand obtained in step 2); and 4) mixing the sense oligonucleotide strand obtained in step 2) and an antisense oligonucleotide strand obtained in step 3) in RNA annealing buffer at a molar ratio of 1:1, heating the mixture, and then naturally cooling the mixture to room temperature, so that a double-stranded saRNA is obtained, wherein the nucleic acid sequence of the human p21 promoter is selected from a group consisting of SEQ ID NOs: 5, 6, 7, 8, 9, 10, 11, and 12.

In certain embodiments, at least one nucleotide of the saRNA is a chemically modified nucleotide, and the chemical modification is at least one of the following modifications:

(1) modification of a phosphodiester bond connecting nucleotides in the nucleotide sequence of the saRNA;

(2) modification of 2′-OH of ribose in the nucleotide sequence of the saRNA;

(3) modification of a base in the nucleotide sequence of the saRNA; or

(4) at least one nucleotide in the nucleotide sequence of the small activating nucleic acid molecule being a locked nucleic acid.

In certain embodiments, the expression of p21 is activated or upregulated by at least 10%, such as more than 15%, 20%, 30%, 40%, 50%, 80%, 100%, or 200%.

The present invention further provides a use of the saRNA in the preparation of a formulation for activating or upregulating the expression of p21 in a cell. The saRNA is introduced into the cell directly, or is produced in the cell after a nucleotide sequence encoding the saRNA is introduced into the cell. The cell is a mammal cell, preferably a human cell, and more preferably a human tumor cell. The human cell can be an isolated human cell line or can be present in a human body.

In certain embodiments, the human body is a patient with a tumor caused by insufficient p21 protein expression, wherein administration of an effective amount of the small activating nucleic acid molecule can treat the tumor, and the tumor is preferably a bladder cancer, a prostate cancer, a hepatocellular carcinoma, or a colorectal cancer.

In another aspect, the present invention further provides an isolated p21 saRNA target site, wherein the target site is any continuous 16-35 nucleotide sequence selected from the group consisting of SEQ ID NOs. 5-12.

In yet another aspect, the present invention discloses a method for activating or upregulating the expression of human p21 in a cell, wherein the method comprises administrating the saRNA as described in any one of the aforementioned embodiments to a subject or a cell. The saRNA can be introduced into the cell directly, or can be produced in the cell after a nucleotide sequence encoding the saRNA is introduced into the cell. The cell is a mammalian cell, preferably a human cell, more preferably a human tumor cell, and most preferably a bladder cancer cell, a prostate cancer cell, a hepatocellular carcinoma cell, or a colorectal cancer cell.

The present invention further discloses a composition containing the aforementioned saRNA and a pharmaceutically acceptable carrier. The pharmaceutically acceptable carrier is a liposome, a macromolecular polymer, or a polypeptide.

The present invention further discloses a use of the saRNA or the composition as described above in the preparation of a formulation for activating or upregulating p21 expression. Preferably a use of the saRNA or the composition in the preparation of a formulation is for treating a tumor or a benign proliferative lesion. Preferably, the tumor is a bladder cancer, a prostate cancer, a hepatocellular carcinoma, or a colorectal cancer.

### BRIEF DESCRIPTION OF THE DRAWINGS

FIG. 1 shows the sequence of the p21 gene promoter, from −1000 bp upstream of the transcription start site (TSS) to +3 bp downstream of the TSS (SEQ ID NO: 469). The TSS is represented by a bent arrow.

FIGS. 2A and 2B show saRNA hotspot regions in the p21 promoter as revealed by screening. According to the sequence of the p21 promoter shown in FIG. 1, four hundred and thirty-nine (439) double-stranded RNA molecules were designed, chemically synthesized, and each double-stranded RNA molecule was transfected into PC3 human prostate cancer cells. 72 hours after transfection, the mRNA levels of p21 were determined by using QuantiGene 2.0 assay. FIG. 2A shows the fold change (Y-axis) of p21 mRNA levels caused by each of the 439 double-stranded RNA molecules (X-axis) relative to a control treatment (Mock). The double-stranded RNA molecules on the X-axis were sorted according to their positions relative to the TSS of p21 (from the most upstream RAG-898 to the most downstream RAG-177). Eight hotspot regions were identified and shown (grayish rectangular boxes). FIG. 2B shows the data of FIG. 2A by sorting the double-stranded RNA molecules by their activity in activating p21 mRNA expression in ascending order. The dotted lines in FIG. 2A and FIG. 2B represent the two-fold induction.

FIGS. 3A-3H show the activating effects of the double-stranded RNA molecules targeting the hotspot regions 1 to 8 on the p21 promoter (FIG. 3A: hotspot region 1; FIG. 3B: hotspot region 2; FIG. 3C: hotspot region 3; FIG. 3D: hotspot region 4; FIG. 3E: hotspot region 5; FIG. 3F: hotspot region 6; FIG. 3G: hotspot region 7; FIG. 3H: hotspot region 8).

FIGS. 4A and 4B show the mRNA levels of p21 assessed by RT-qPCR for verifying the result obtained from QuantiGene 2.0 assay. FIG. 4A shows the p21 mRNA level determined by RT-qPCR. The 439 double-stranded RNA molecules were divided into four groups according to their activities in inducing p21 mRNA expression from the highest to the lowest, and 5 double-stranded RNA molecules were randomly selected from each group and individually transfected into PC3 cells at a concentration of 10 nM. 72 hours after transfection, total cellular RNA was extracted from the transfected cells and reverse transcribed into cDNA, which was amplified by RT-qPCR to determine p21 mRNA level. FIG. 4B shows the correlation of relative p21 mRNA level induced by the double-stranded RNA molecules as determined by QuantiGene 2.0 (X-axis) and by RT-qPCR (Y-axis).

FIGS. 5A-5C show the effect of the saRNAs in inducing the mRNA expression of p21 and suppressing the proliferation of KU-7 cells. KU-7 cells were transfected with each of three saRNAs (RAG-431, RAG-553, or RAG-688) at a concentration of 10 nM for 72 hours. FIG. 5A shows the mRNA expression levels of p21 determined by RT-qPCR. FIG. 5B shows the viability of saRNA treated cells as evaluated by the CCK-8 assay and plotted as percentages relative to the cell viability for control treatment (Mock). FIG. 5C shows representative phase-contrast cell images (100×) at the end of transfection.

FIGS. 6A-6C show the effect of the saRNAs in inducing the mRNA expression of p21 and suppressing the proliferation of HCT116 cells. HCT116 cells were transfected with each of the three saRNAs (RAG-431, RAG-553, or RAG-688) at a concentration of 10 nM for 72 hours. FIG. 6A shows the mRNA expression levels of p21 determined by RT-qPCR. FIG. 6B shows the cell viability as evaluated by the CCK-8 assay and plotted as percentages relative to the cell viability for control treatment (Mock). FIG. 6C shows representative phase-contrast cell images (100×) at the end of transfection.

FIGS. 7A-7C show the effect of the saRNAs in inducing the mRNA expression of p21 and suppressing the proliferation of HepG2 cells. HepG2 cells were transfected with each of the three saRNAs (RAG-431, RAG-553, or RAG-688) at a concentration of 10 nM for 72 hours. FIG. 7A shows the mRNA expression levels of p21 determined by RT-qPCR. FIG. 7B shows the cell viability as evaluated by the CCK-8 assay and plotted as percentages relative to the cell viability for control treatment (Mock). FIG. 7C shows representative phase-contrast cell images (100×) at the end of transfection.

### DETAILED DESCRIPTION OF THE INVENTION

The present invention is further described hereinafter by specific description.

Unless otherwise defined, all the technological and scientific terms used herein have the same meanings as those generally understood by those of ordinary skill in the art covering the present invention.

In the present application, singular forms, such as “a” and “this”, include plural objects, unless otherwise specified clearly in the context.

### Definition

The term “complementary” as used herein refers to the capability of forming base pairs between two oligonucleotide strands. The base pairs are generally formed by hydrogen bonds between nucleotide units in the antiparallel oligonucleotide strands. The bases of the complementary oligonucleotide strands can be paired in the Watson-Crick manner (such as A to T, A to U, and C to G) or in any other manner allowing the formation of a duplex (such as Hoogsteen or reverse Hoogsteen base pairing). “100% pairing” or “complete complementarity” refers to 100% complementarity, that is, all the nucleotide units of the two strands are bound by hydrogen bonds.

“Complete complementarity” or “100% pairing” means that each nucleotide unit from the first oligonucleotide strand can form a hydrogen bond with the second oligonucleotide strand in the double-stranded region of the double-stranded oligonucleotide molecule, with no base pair being “mispaired”. “Incomplete complementarity” means that not all the nucleotide units of the two strands are bound with each other by hydrogen bonds. For example, for two oligonucleotide strands each of 20 nucleotides in length in the double-stranded region, if only two base pairs in this double-stranded region can be formed through hydrogen bonds, the oligonucleotide strands have a complementarity of 10%. In the same example, if 18 base pairs in this double-stranded region can be formed through hydrogen bonds, the oligonucleotide strands have a complementarity of 90%. “Substantial complementarity” refers to more than about 79%, about 80%, about 85%, about 90%, or about 95% complementarity.

The term “oligonucleotide” as used herein refers to polymers of nucleotides, and includes, but is not limited to, single-stranded or double-stranded molecules of DNA, RNA, DNA/RNA hybrid, oligonucleotide strands containing regularly and irregularly alternating deoxyribosyl portions and ribosyl portions, as well as modified and naturally or unnaturally existing frameworks for such oligonucleotides.

The term “oligoribonucleotide” as used herein refers to an oligonucleotide containing two or more modified or unmodified ribonucleotides and/or analogues thereof.

The terms “oligonucleotide strand” and “oligonucleotide sequence” as used herein can be used interchangeably, referring to a generic term for short nucleotide sequences having less than 50 bases (the nucleic acid can be deoxyribonucleic acid (DNA) or ribonucleic acid (RNA)). In the present invention, the length of an oligonucleotide strand can be any length from 17 to 30 nucleotides.

The term “gene” as used herein refers to all nucleotide sequences required to encode a polypeptide chain or to transcribe a functional RNA. “Gene” can be an endogenous or fully or partially recombinant gene for a host cell (for example, because an exogenous oligonucleotide and a coding sequence for coding a promoter are introduced into a host cell, or a heterogeneous promoter adjacent to an endogenous coding sequence is introduced into a host cell). For example, the term “gene” includes a nucleic acid sequence composed of exons and introns. Protein-coding sequences are, for example, sequences contained within exons in an open reading frame between an initiation codon and a termination codon, and as used herein, “gene” can comprise a gene regulatory sequence, such as a promoter, an enhancer, and all other sequences known in the art for controlling the transcription, expression or activity of another gene, no matter whether the gene contains a coding sequence or a non-coding sequence. In one case, for example, “gene” can be used to describe a functional nucleic acid containing a regulatory sequence such as a promoter or an enhancer. The expression of a recombinant gene can be controlled by one or more types of heterogenous regulatory sequences.

The term “target gene” as used herein can refer to nucleic acid sequences, transgenes, viral or bacterial sequences, chromosomes or extrachromosomal genes that are naturally present in organisms, and/or can be transiently or stably transfected or incorporated into cells and/or chromatins thereof. The target gene can be a protein-coding gene or a non-protein-coding gene (such as microRNA gene and long non-coding RNA gene). The target gene generally contains a promoter sequence, and the positive regulation for the target gene can be achieved by designing a saRNA having sequence homology with the promoter sequence, characterized as the upregulation of expression of the target gene. “Sequence of a target gene promoter” refers to a non-coding sequence of the target gene, and the reference of the sequence of a target gene promoter in the phrase “complementary with the sequence of a target gene promoter” of the present invention means a coding strand of the sequence, also known as a non-template strand, i.e. a nucleic acid sequence having the same sequence as the coding sequence of the gene. “Target sequence” refers to a sequence fragment in the target gene promoter sequence, which is homologous or complementary with a sense oligonucleotide strand or an antisense oligonucleotide strand of a saRNA.

As used herein, the terms “sense strand” and “sense oligonucleotide strand” can be used interchangeably, and the sense oligonucleotide strand of a saRNA refers to a first ribonucleic acid strand having homology with the coding strand of the promoter sequence of the target gene in the saRNA duplex.

As used herein, the terms “antisense strand” and “antisense oligonucleotide strand” can be used interchangeably, and the antisense oligonucleotide strand of a saRNA refers to a second ribonucleic acid strand complementary with the sense oligonucleotide strand in the saRNA duplex.

The term “coding strand” as used herein refers to a DNA strand in the target gene which cannot be used for transcription, and the nucleotide sequence of this strand is the same as that of RNA produced from transcription (in the RNA, T in DNA is replaced by U). The coding strand of the double-stranded DNA sequence of the target gene promoter described in the present invention refers to a promoter sequence on the same DNA strand as the DNA coding strand of the target gene.

The term “template strand” as used herein refers to the other strand complementary with the coding strand in the double-stranded DNA of the target gene, i.e. the strand that, as a template, can be transcribed into RNA, and this strand is complementary with the transcribed RNA (A to U, G to C). In the process of transcription, RNA polymerase is bound with the template strand, moves along the 3′→5′ direction of the template strand, and catalyzes the synthesis of the RNA along the 5′→3′ direction. The template strand of the double-stranded DNA sequence of the target gene promoter described in the present invention refers to a promoter sequence on the same DNA strand as the DNA template strand of the target gene.

The term “promoter” as used herein refers to a nucleic acid sequence, which does not encode a protein, which plays a regulatory role for the transcription of a protein-coding or RNA-coding nucleic acid sequence by associating with them spatially. Generally, a eukaryotic promoter contains 100 to 5,000 base pairs, although this length range is not intended to limit the term “promoter” as used herein. Although the promoter sequence is generally located at the 5′ terminus of a protein-coding or RNA-coding sequence, in some cases, the promoter sequence also exists in exon and intron sequences.

The term “transcription start site” as used herein refers to a nucleotide marking the transcription start on the template strand of a gene. The transcription start site can appear on the template strand of the promoter region. A gene can have more than one transcription start site.

The term “sequence identity” or “sequence homology” as used herein means that one oligonucleotide strand (sense or antisense) of a saRNA has at least 75% similarity with a region on the coding strand or template strand of the promoter sequence of a target gene.

The term “overhang” as used herein refers to non-base-paired nucleotides at the terminus (5′ or 3′) of an oligonucleotide strand, which is formed by one strand extending out of the other strand in a duplex oligonucleotide. A single-stranded region extending out of the 3′ terminus and/or 5′ terminus of a duplex is referred to as an overhang.

As used herein, the terms “gene activation” or “activating gene expression” can be used interchangeably, and means an increase or upregulation in transcription, translation, expression or activity of a certain nucleic acid as determined by measuring the transcription level, mRNA level, protein level, enzymatic activity, methylation state, chromatin state or configuration, translation level or the activity or state in a cell or biological system of a gene. These activities or states can be determined directly or indirectly. In addition, “gene activation” or “activating gene expression” refers to an increase in activity associated with a nucleic acid sequence, regardless the mechanism of such activation. For example, gene activation occurs at the transcriptional level to increase transcription into RNA and the RNA is translated into a protein, thereby increasing the expression of the protein.

As used herein, the terms “small activating RNA,” “saRNA,” and “small activating nucleic acid molecule” can be used interchangeably, and refer to a ribonucleic acid molecule that can upregulate target gene expression. The saRNA can be composed of a first ribonucleic acid strand (antisense strand, also referred to as antisense oligonucleotide strand) containing a ribonucleotide sequence having sequence homology with the non-coding nucleic acid sequence (e.g., a promotor and an enhancer) of a target gene and a second ribonucleic acid strand (sense strand, also referred to as sense oligonucleotide strand) containing a nucleotide sequence complementary with the first ribonucleic acid strand, wherein the first ribonucleic acid strand and the second ribonucleic acid strand form a duplex. The saRNA can also be comprised of a synthesized or vector-expressed single-stranded RNA molecule that can form a hairpin structure by two complementary regions within the molecule, wherein the first region contains a nucleic acid sequence having sequence homology with the target sequence of a promoter of a gene, and a nucleic acid sequence contained in the second region is complementary with the first region. The length of the duplex region of the saRNA molecule is typically about 10 to about 50, about 12 to about 48, about 14 to about 46, about 16 to about 44, about 18 to about 42, about 20 to about 40, about 22 to about 38, about 24 to about 36, about 26 to about 34, and about 28 to about 32 base pairs, and typically about 10, about 15, about 20, about 25, about 30, about 35, about 40, about 45, or about 50 base pairs. In addition, the terms “saRNA”, “small activating RNA”, and “small activating nucleic acid molecule” also contain nucleic acids other than the ribonucleotide, including, but not limited to, modified nucleotides or analogues.

As used herein, the term “hotspot” refers to a promoter region of a gene where targets for functional saRNAs are enriched. In these hotspot regions, at least 60% of the small activating nucleic acid molecules targeting these hotspot regions can induce a 1.5-fold or more change in the mRNA expression of a target gene.

As used herein, the term “p21” refers to the p21^{WAF1/CIP1} gene, also known as the CDKN1A gene. As a cyclin-dependent kinase (CDK) inhibitor, p21 is an important tumor suppressor gene, also known as “target gene” sometimes. Overexpression of p21 or activation of endogenous p21 transcription has been shown to inhibit the growth of cultured tumor cells and tumors in vivo.

As used herein, the term “synthesis” refers to a method for synthesis of an oligonucleotide, including any method allowing RNA synthesis, such as chemical synthesis, in-vitro transcription, and/or vector-based expression. The present invention provides a method for preparing the small activating nucleic acid molecule, which comprises sequence design and sequence synthesis. The synthesis of the sequence of the small activating nucleic acid molecule can adopt a chemical synthesis or can be entrusted to a biotechnology company specialized in nucleic acid synthesis. Generally speaking, the chemical synthesis comprises the following four steps: (1) synthesis of oligomeric ribonucleotides; (2) deprotection; (3) purification and isolation; (4) desalination and annealing. For example, the specific steps for chemically synthesizing the double-stranded RNA molecule of the present invention, such as saRNA, are as follows:

(1) Synthesis of Oligomeric Ribonucleotides

Synthesis of 1 micromole of RNA was set in an automatic DNA/RNA synthesizer (e.g., Applied Biosystems EXPEDITE8909), and the coupling time of each cycle was also set as 10 to 15 minutes. With a solid phase-bonded 5′-O-p-dimethoxytriphenylmethyl-thymidine substrate as an initiator, one base was bonded to the solid phase substrate in the first cycle, and then, in the nth (19≥n≥2) cycle, one base was bonded to the base bonded in the n−1 cycle. This process was repeated until the synthesis of the whole nucleic acid sequence was completed.

(2) Deprotection

The solid phase substrate bonded with the saRNA was put into a test tube, and 1 ml of a solution of the mixture of ethanol and ammonium hydroxide (volume ratio: 1:3) was added into the test tube. The test tube was then sealed and placed in an incubator, and the mixture was incubated at 25-70° C. for 2 to 30 hours. The solution containing the solid phase substrate bonded with the saRNA was filtered, and the filtrate was collected. The solid phase substrate was rinsed with double distilled water twice (1 ml each time), and the filtrate was collected. The eluents were combined and collected, and dried under vacuum for 1 to 12 hours. Then, 1 ml of a solution of tetrabutylammonium fluoride in tetrahydrofuran (1 M) was added. After 4 to 12 hours of standing at room temperature, 2 ml of n-butanol was then added. Precipitate was collected to obtain a single-stranded crude product of saRNA by high-speed centrifugation.

(3) Purification and Isolation

The obtained crude product of saRNA was dissolved in 2 ml of aqueous ammonium acetate solution with a concentration of 1 mol/ml, and the solution was separated by a reversed-phase C18 column of high pressure liquid chromatography to obtain a purified single-stranded product of saRNA.

(4) Desalination and Annealing

Salts were removed by gel filtration (size exclusion chromatography). A single sense oligomeric ribonucleic acid strand and a single antisense oligomeric ribonucleic acid strand were mixed into 1 to 2 ml of buffer (10 mM Tris, pH=7.5-8.0, 50 mM NaCl) at a molar ratio of 1:1. The solution was heated to 95° C., and was then slowly cooled to room temperature to obtain a solution containing saRNA.

Materials and Methods

Cell Culture and Transfection

Cell lines RT4, KU-7, T24, J82, TCCSUP, and HT-1197 were cultured in the modified McCoy's 5A medium (Gibco); cell lines 5637, PC3, and Bel-7402 were cultured in RPMI1640 medium (Gibco); and the cell line UM-UC-3 was cultured in basal medium (Gibco). All the media contained 10% bovine calf serum (Sigma-Aldrich) and 1% penicillin/streptomycin (Gibco). The cells were cultured at 5% CO₂ and 37° C. Double-stranded RNA molecules designed in the experiment were transfected into cells using RNAiMax (Invitrogen, Carlsbad, Calif.) according to the instructions provided by the manufacturer at the concentration of 10 nM (unless otherwise specified).

### RNA Isolation and Reverse Transcription-Polymerase Chain Reaction

Cells were seeded into a 6-well plate with 2-3×10⁵ cells/well and were reverse transfected with oligonucleotide duplexes. At the end of the transfection, total cellular RNA was isolated using an RNeasy Plus Mini kit (Qiagen; Hilden, Germany) according to its manual. The isolated RNA (1 μg) was reverse transcribed into cDNA by using a PrimeScript RT kit containing gDNA Eraser (Takara, Shlga, Japan). The resulted cDNA was amplified in an ABI 7500 Fast Real-time PCR System (Applied Biosystems; Foster City, Calif.) using SYBR Premix Ex Taq II (Takara, Shlga, Japan) reagents and target gene specific primers. The reaction conditions were: 95° C. for 3 seconds, 60° C. for 30 seconds, and 40 cycles. Amplification of GAPDH served as an internal control. All primer sequences are listed in Table 1.

TABLE 1
Primer sequences for RT-qPCR assay

| Primer Title | Sequence No. | Sequence (5′-3′) |
|---|---|---|
| GAPDH F | SEQ ID NO 1 | ATCACCATCTTCCAGGAGCGA |
| GAPDH R | SEQ ID NO 2 | TTCTCCATGGTGGTGAAGACG |
| CDKN1A F | SEQ ID NO 3 | GGAAGACCATGTGGACCTGT |
| CDKN1A R | SEQ ID NO 4 | GGATTAGGGCTTCCTCTTGG |

Assay of Cell Proliferation Cells were plated into a 96-well plate with 2-4×10³ cells/well, cultured overnight, and transfected with oligonucleotide duplexes. Three days after transfection, the CCK8 reagent (Dojindo; Rockville, Md.) was used to assay the cell proliferation according to its manual. Briefly, 10 μL of CCK8 reagent was added into each well on the plate which was then incubated at 37° C. for 1 hour. Absorbance for each well on the plate was measured at 450 nm by a microplate reader.

QuantiGene 2.0 Assay

Cells were plated into a 96-well plate and transfected with oligonucleotide duplexes. 72 hours after transfection, the mRNA levels of target genes were quantitatively assayed with a QuantiGene 2.0 kit (AffyMetrix; Santa Clara, Calif.). QuantiGene 2.0 assay is based on hybridization technology, wherein mRNA levels were directly quantified with gene-specific probes. The experimental procedure is briefly described as follows: a lysis solution was added to lyse the transfected cells, and the cell lysates were added into a capture well plate coated with probe for CDKN1A (p21) and HPRT1 which serves as a housekeeping gene for hybridizing overnight at 55° C. In order to enhance the hybridization signal, hybridizations with 2.0 PreAMP Probe, 2.0 AMP Probe, and 2.0 Label Probe were conducted sequentially in 100 μL of a corresponding buffer solution (provided by Quantigene 2.0 kit). All the hybridizations were conducted with shaking at 50-55° C. for 1 hour. After the last wash step, 2.0 Substrate was added to the solution and incubated at room temperature for 5 minutes. Optical signals were detected with an Infinite 200 PRO plate reader (Tecan, Switzerland).

Statistical Analysis

Results were represented as mean±standard deviation. One-way analysis of variance was carried out with GraphPad Prism software (GraphPad Software), and then a Tukey's t test was conducted for pairwise comparisons. A statistical significance was set as *p<0.05, **p<0.01, and ***p<0.001.

### EXAMPLES

The present invention is further illustrated by the following examples. These examples are provided merely for illustration purposes and shall not be interpreted to limit the scope or content of the present invention in any way.

### Example 1: Screening of Functional saRNAs Targeting the Promoter Region of p21

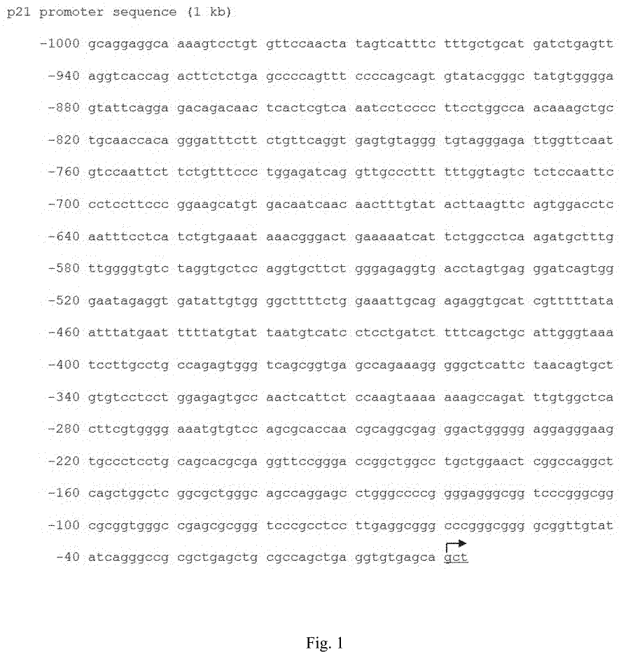

The 1 kb promoter sequence (FIG. 1) (SEQ ID NO:469) of p21 was retrieved from the UCSC Genome database to screen for functional saRNAs capable of activating p21 gene expression. A total of 982 target sequences were obtained by selecting a target with a size of 19 bp starting from the −1 kb position upstream of TSS and moving toward the TSS one base pair (bp) at a time. The target sequences were filtered to remove those that have a GC content higher than 65% or lower than 35% and those that contain 5 or more consecutive nucleotides. After filtration of the target sequences, 439 target sequences remained and were used as candidates for screening. Corresponding double-stranded RNA molecules were chemically synthesized based on these candidate sequences. Each of the sense strand and antisense strand in the double-stranded RNA molecule used in the experiment had 21 nucleotides in length. The 19 nucleotides in the 5′ region of the first ribonucleic acid strand (sense strand) of the double-stranded RNA molecule (e.g., double-stranded saRNA) had 100% homology with the target sequence of the promoter, and the 3′ terminus of the first ribonucleic acid strand contained a dTdT overhang. The 19 nucleotides in the 5′ region of the second ribonucleic acid strand were fully complementary with the 19 nucleotides in the 5′ region sequence of the first ribonucleic acid strand, and the 3′ terminus of the second ribonucleic acid strand contained a dTdT overhang.

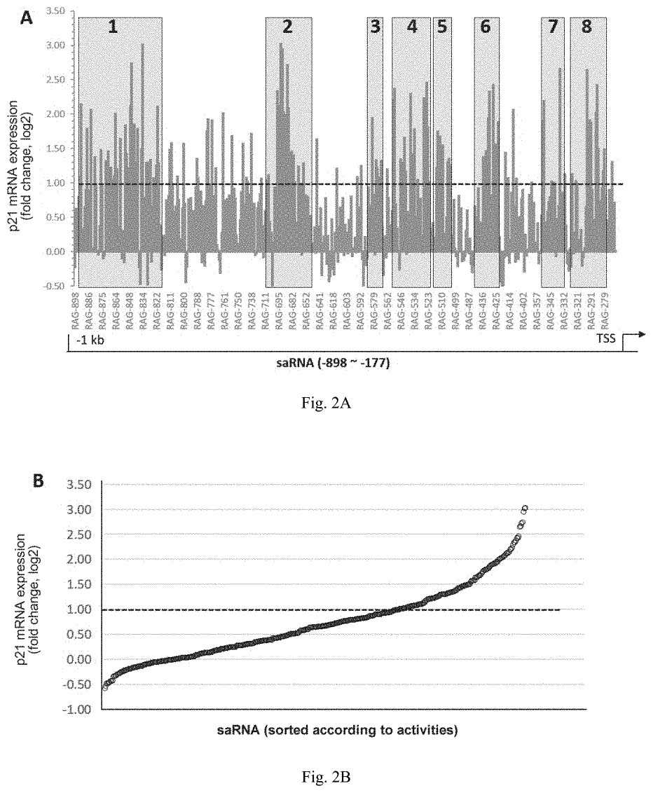

The aforementioned double-stranded RNA molecules were transfected into PC3 prostate cancer cells at a final concentration of 10 nM, and 72 hours after transfection, the p21 mRNA level was detected with the QuantiGene 2.0 kit. The fold change of p21 mRNA level induced by each double-stranded RNA molecule relative to the blank control was calculated and plotted in FIG. 2. In this study, the fold changes of p21 mRNA level induced by all double-stranded RNA molecules ranged from 0.66 (suppression) to 8.12 (induction) (FIG. 2B). There were 361 (82.2%) double-stranded RNA molecules inducing a 1.01-fold to 8.12-fold change of p21 expression, 74 (16.9%) double-stranded RNA molecules exhibiting a suppressing effect (0.99-fold to 0.66-fold change), and 4 (0.9%) double-stranded RNA molecules having no influence on p21 mRNA level (1.0-fold change).

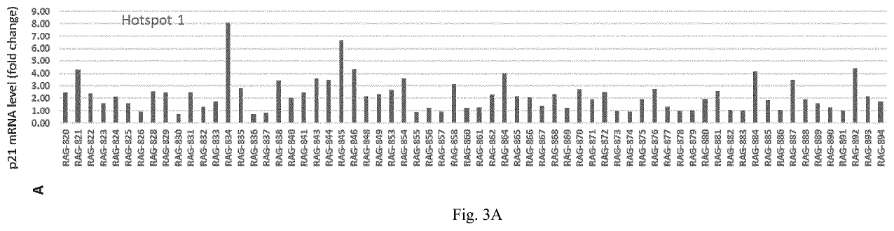

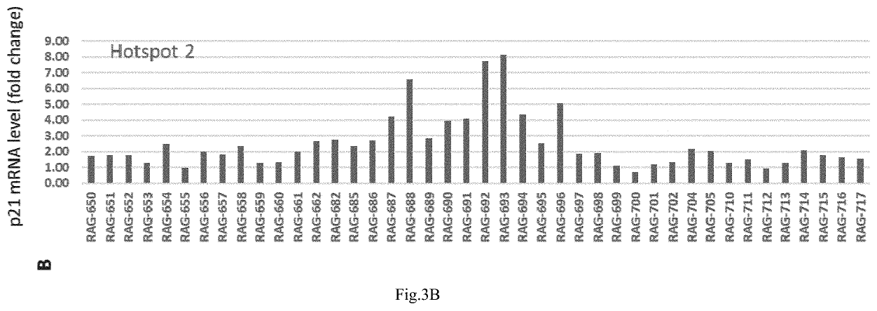

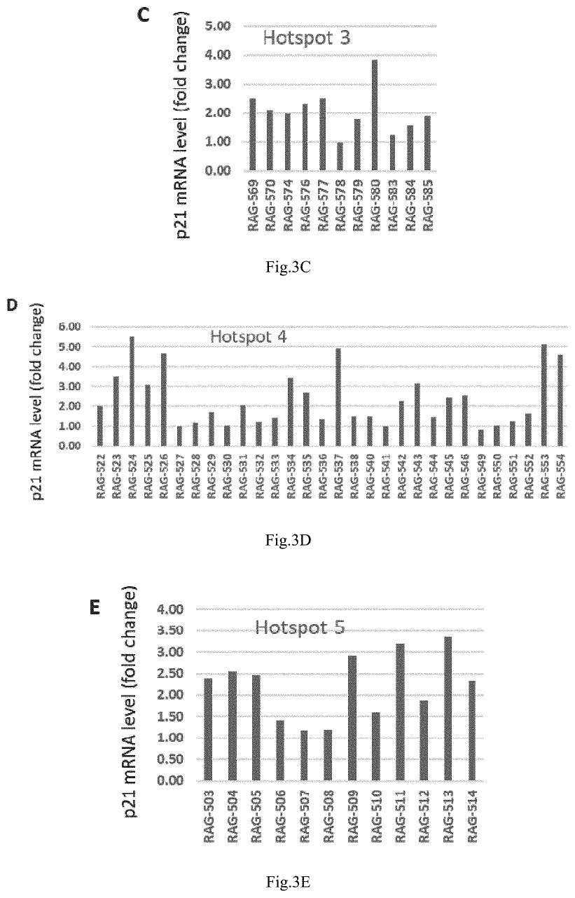

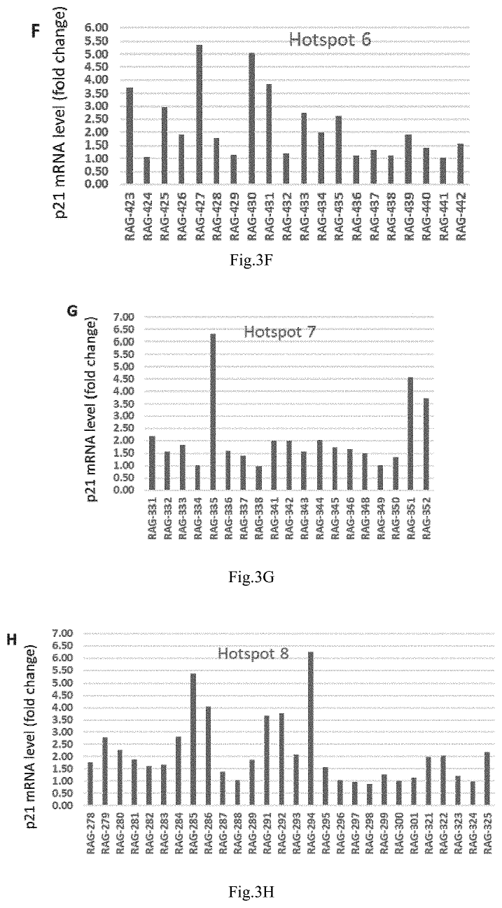

Among the 439 screened double-stranded RNA molecules, 132 double-stranded RNA molecules (30.1%) could induce at least a 2-fold change in p21 mRNA level, and 229 (52.4%) double-stranded RNA molecules could induce at least a 1.5-fold change in p21 mRNA level. These double-stranded RNA molecules that upregulated p21 mRNA expression by more than 10% were functional saRNAs. The targets for these functional saRNAs were scattered in the entire p21 promoter region. However, 8 discrete regions showed enrichment for saRNA targets and these regions are considered as saRNA “hotspots.” The hotspot is defined as a region containing at least 10 corresponding saRNAs, wherein at least 60% of them could induce a 1.5-fold or more change in p21 mRNA expression (FIG. 2A and FIG. 3). The target sequences for hotspots 1-8 and corresponding saRNA sequences are listed in Table 2 and Table 3 respectively.

TABLE 2
Target sequences of hotspot regions of p21 promoter

| | | | Location | |
|---|---|---|---|---|
| | | | (relative to | Length |
| Hotspot | Sequence No. | DNA Sequence (5′-3′) | TSS) | (bp) |
| Hotspot 1 | SEQ ID NO: 5 | ggctatgtggggagtattcaggagacagacaactcactcgt | −893~−801 | 92 |
| | | caaatcctccccttcctggccaacaaagctgctgcaaccac | | |
| | | agggatttct | | |
| Hotspot 2 | SEQ ID NO: 6 | ggtagtctctccaattccctccttcccggaagcatgtgacaat | −717~−632 | 86 |
| | | caacaactttgtatacttaagttcagtggacctcaatttcctc | | |
| Hotspot 3 | SEQ ID NO: 7 | ctttgttggggtgtctaggtgctccaggtgcttct | −585~−551 | 35 |
| | | ttctgggagaggtgacctagtgagggatcagtgggaataga | | |
| Hotspot 4 | SEQ ID NO: 8 | ggtgatattg | −554~−504 | 51 |
| Hotspot 5 | SEQ ID NO: 9 | aggtgatattgtggggcttttctggaaatt | −514~−485 | 30 |
| Hotspot 6 | SEQ ID NO: 10 | attaatgtcatcctcctgatcttttcagctgcattggg | −442~−405 | 38 |
| Hotspot 7 | SEQ ID NO: 11 | tctaacagtgctgtgtcctcctggagagtgccaactcatt | −352~−313 | 40 |
| Hotspot 8 | SEQ ID NO: 12 | gtgccaactcattctccaagtaaaaaaagccagatttgtggct | −325~−260 | 66 |
| | | cacttcgtggggaaatgtgtcca | | |

TABLE 3
Screened double-stranded RNA molecules located in hotspot regions
of p21 promoter

| Title | Sequence No. | Sequence (5′-3′) | Length |
|---|---|---|---|
| Hotspot 8 | | | |
| RAG-278 | SEQ ID NO: 13 | UCGUGGGGAAAUGUGUCCA[dT][dT] | 21 nt |
| | SEQ ID NO: 14 | UGGACACAUUUCCCCACGA[dT][dT] | 21 nt |
| RAG-279 | SEQ ID NO: 15 | UUCGUGGGGAAAUGUGUCC[dT][dT] | 21 nt |
| | SEQ ID NO: 16 | GGACACAUUUCCCCACGAA[dT][dT] | 21 nt |
| RAG-280 | SEQ ID NO: 17 | CUUCGUGGGGAAAUGUGUC[dT][dT] | 21 nt |
| | SEQ ID NO: 18 | GACACAUUUCCCCACGAAG[dT][dT] | 21 nt |
| RAG-281 | SEQ ID NO: 19 | ACUUCGUGGGGAAAUGUGU[dT][dT] | 21 nt |
| | SEQ ID NO: 20 | ACACAUUUCCCCACGAAGU[dT][dT] | 21 nt |
| RAG-282 | SEQ ID NO: 21 | CACUUCGUGGGGAAAUGUG[dT][dT] | 21 nt |
| | SEQ ID NO: 22 | CACAUUUCCCCACGAAGUG[dT][dT] | 21 nt |
| RAG-283 | SEQ ID NO: 23 | UCACUUCGUGGGGAAAUGU[dT][dT] | 21 nt |
| | SEQ ID NO: 24 | ACAUUUCCCCACGAAGUGA[dT][dT] | 21 nt |
| RAG-284 | SEQ ID NO: 25 | CUCACUUCGUGGGGAAAUG[dT][dT] | 21 nt |
| | SEQ ID NO: 26 | CAUUUCCCCACGAAGUGAG[dT][dT] | 21 nt |
| RAG-285 | SEQ ID NO: 27 | GCUCACUUCGUGGGGAAAU[dT][dT] | 21 nt |
| | SEQ ID NO: 28 | AUUUCCCCACGAAGUGAGC[dT][dT] | 21 nt |
| RAG-286 | SEQ ID NO: 29 | GGCUCACUUCGUGGGGAAA[dT][dT] | 21 nt |
| | SEQ ID NO: 30 | UUUCCCCACGAAGUGAGCC[dT][dT] | 21 nt |
| RAG-287 | SEQ ID NO: 31 | UGGCUCACUUCGUGGGGAA[dT][dT] | 21 nt |
| | SEQ ID NO: 32 | UUCCCCACGAAGUGAGCCA[dT][dT] | 21 nt |
| RAG-288 | SEQ ID NO: 33 | GUGGCUCACUUCGUGGGGA[dT][dT] | 21 nt |
| | SEQ ID NO: 34 | UCCCCACGAAGUGAGCCAC[dT][dT] | 21 nt |
| RAG-289 | SEQ ID NO: 35 | UGUGGCUCACUUCGUGGGG[dT][dT] | 21 nt |
| | SEQ ID NO: 36 | CCCCACGAAGUGAGCCACA[dT][dT] | 21 nt |
| RAG-291 | SEQ ID NO: 37 | UUUGUGGCUCACUUCGUGG[dT][dT] | 21 nt |
| | SEQ ID NO: 38 | CCACGAAGUGAGCCACAAA[dT][dT] | 21 nt |
| RAG-292 | SEQ ID NO: 39 | AUUUGUGGCUCACUUCGUG[dT][dT] | 21 nt |
| | SEQ ID NO: 40 | CACGAAGUGAGCCACAAAU[dT][dT] | 21 nt |
| RAG-293 | SEQ ID NO: 41 | GAUUUGUGGCUCACUUCGU[dT][dT] | 21 nt |
| | SEQ ID NO: 42 | ACGAAGUGAGCCACAAAUC[dT][dT] | 21 nt |
| RAG-294 | SEQ ID NO: 43 | AGAUUUGUGGCUCACUUCG[dT][dT] | 21 nt |
| | SEQ ID NO: 44 | CGAAGUGAGCCACAAAUCU[dT][dT] | 21 nt |
| RAG-295 | SEQ ID NO: 45 | CAGAUUUGUGGCUCACUUC[dT][dT] | 21 nt |
| | SEQ ID NO: 46 | GAAGUGAGCCACAAAUCUG[dT][dT] | 21 nt |
| RAG-296 | SEQ ID NO: 47 | CCAGAUUUGUGGCUCACUU[dT][dT] | 21 nt |
| | SEQ ID NO: 48 | AAGUGAGCCACAAAUCUGG[dT][dT] | 21 nt |
| RAG-297 | SEQ ID NO: 49 | GCCAGAUUUGUGGCUCACU[dT][dT] | 21 nt |
| | SEQ ID NO: 50 | AGUGAGCCACAAAUCUGGC[dT][dT] | 21 nt |
| RAG-298 | SEQ ID NO: 51 | AGCCAGAUUUGUGGCUCAC[dT][dT] | 21 nt |
| | SEQ ID NO: 52 | GUGAGCCACAAAUCUGGCU[dT][dT] | 21 nt |
| RAG-299 | SEQ ID NO: 53 | AAGCCAGAUUUGUGGCUCA[dT][dT] | 21 nt |
| | SEQ ID NO: 54 | UGAGCCACAAAUCUGGCUU[dT][dT] | 21 nt |
| RAG-300 | SEQ ID NO: 55 | AAAGCCAGAUUUGUGGCUC[dT][dT] | 21 nt |
| | SEQ ID NO: 56 | GAGCCACAAAUCUGGCUUU[dT][dT] | 21 nt |
| RAG-301 | SEQ ID NO: 57 | AAAAGCCAGAUUUGUGGCU[dT][dT] | 21 nt |
| | SEQ ID NO: 58 | AGCCACAAAUCUGGCUUUU[dT][dT] | 21 nt |
| RAG-321 | SEQ ID NO: 59 | CAACUCAUUCUCCAAGUAA[dT][dT] | 21 nt |
| | SEQ ID NO: 60 | UUACUUGGAGAAUGAGUUG[dT][dT] | 21 nt |
| RAG-322 | SEQ ID NO: 61 | CCAACUCAUUCUCCAAGUA[dT][dT] | 21 nt |
| | SEQ ID NO: 62 | UACUUGGAGAAUGAGUUGG[dT][dT] | 21 nt |
| RAG-323 | SEQ ID NO: 63 | GCCAACUCAUUCUCCAAGU[dT][dT] | 21 nt |
| | SEQ ID NO: 64 | ACUUGGAGAAUGAGUUGGC[dT][dT] | 21 nt |
| RAG-324 | SEQ ID NO: 65 | UGCCAACUCAUUCUCCAAG[dT][dT] | 21 nt |
| | SEQ ID NO: 66 | CUUGGAGAAUGAGUUGGCA[dT][dT] | 21 nt |
| RAG-325 | SEQ ID NO: 67 | GUGCCAACUCAUUCUCCAA[dT][dT] | 21 nt |
| | SEQ ID NO: 68 | UUGGAGAAUGAGUUGGCAC[dT][dT] | 21 nt |
| Hotspot 7 | | | |
| RAG-331 | SEQ ID NO: 69 | UGGAGAGUGCCAACUCAUU[dT][dT] | 21 nt |
| | SEQ ID NO: 70 | AAUGAGUUGGCACUCUCCA[dT][dT] | 21 nt |
| RAG-332 | SEQ ID NO: 71 | CUGGAGAGUGCCAACUCAU[dT][dT] | 21 nt |
| | SEQ ID NO: 72 | AUGAGUUGGCACUCUCCAG[dT][dT] | 21 nt |
| RAG-333 | SEQ ID NO: 73 | CCUGGAGAGUGCCAACUCA[dT][dT] | 21 nt |
| | SEQ ID NO: 74 | UGAGUUGGCACUCUCCAGG[dT][dT] | 21 nt |
| RAG-334 | SEQ ID NO: 75 | UCCUGGAGAGUGCCAACUC[dT][dT] | 21 nt |
| | SEQ ID NO: 76 | GAGUUGGCACUCUCCAGGA[dT][dT] | 21 nt |
| RAG-335 | SEQ ID NO: 77 | CUCCUGGAGAGUGCCAACU[dT][dT] | 21 nt |
| | SEQ ID NO: 78 | AGUUGGCACUCUCCAGGAG[dT][dT] | 21 nt |
| RAG-336 | SEQ ID NO: 79 | CCUCCUGGAGAGUGCCAAC[dT][dT] | 21 nt |
| | SEQ ID NO: 80 | GUUGGCACUCUCCAGGAGG[dT][dT] | 21 nt |
| RAG-337 | SEQ ID NO: 81 | UCCUCCUGGAGAGUGCCAA[dT][dT] | 21 nt |
| | SEQ ID NO: 82 | UUGGCACUCUCCAGGAGGA[dT][dT] | 21 nt |
| RAG-338 | SEQ ID NO: 83 | GUCCUCCUGGAGAGUGCCA[dT][dT] | 21 nt |
| | SEQ ID NO: 84 | UGGCACUCUCCAGGAGGAC[dT][dT] | 21 nt |
| RAG-341 | SEQ ID NO: 85 | UGUGUCCUCCUGGAGAGUG[dT][dT] | 21 nt |
| | SEQ ID NO: 86 | CACUCUCCAGGAGGACACA[dT][dT] | 21 nt |
| RAG-342 | SEQ ID NO: 87 | CUGUGUCCUCCUGGAGAGU[dT][dT] | 21 nt |
| | SEQ ID NO: 88 | ACUCUCCAGGAGGACACAG[dT][dT] | 21 nt |
| RAG-343 | SEQ ID NO: 89 | GCUGUGUCCUCCUGGAGAG[dT][dT] | 21 nt |
| | SEQ ID NO: 90 | CUCUCCAGGAGGACACAGC[dT][dT] | 21 nt |
| RAG-344 | SEQ ID NO: 91 | UGCUGUGUCCUCCUGGAGA[dT][dT] | 21 nt |
| | SEQ ID NO: 92 | UCUCCAGGAGGACACAGCA[dT][dT] | 21 nt |
| RAG-345 | SEQ ID NO: 93 | GUGCUGUGUCCUCCUGGAG[dT][dT] | 21 nt |
| | SEQ ID NO: 94 | CUCCAGGAGGACACAGCAC[dT][dT] | 21 nt |
| RAG-346 | SEQ ID NO: 95 | AGUGCUGUGUCCUCCUGGA[dT][dT] | 21 nt |
| | SEQ ID NO: 96 | UCCAGGAGGACACAGCACU[dT][dT] | 21 nt |
| RAG-348 | SEQ ID NO: 97 | ACAGUGCUGUGUCCUCCUG[dT][dT] | 21 nt |
| | SEQ ID NO: 98 | CAGGAGGACACAGCACUGU[dT][dT] | 21 nt |
| RAG-349 | SEQ ID NO: 99 | AACAGUGCUGUGUCCUCCU[dT][dT] | 21 nt |
| | SEQ ID NO: 100 | AGGAGGACACAGCACUGUU[dT][dT] | 21 nt |
| RAG-350 | SEQ ID NO: 101 | UAACAGUGCUGUGUCCUCC[dT][dT] | 21 nt |
| | SEQ ID NO: 102 | GGAGGACACAGCACUGUUA[dT][dT] | 21 nt |
| RAG-351 | SEQ ID NO: 103 | CUAACAGUGCUGUGUCCUC[dT][dT] | 21 nt |
| | SEQ ID NO: 104 | GAGGACACAGCACUGUUAG[dT][dT] | 21 nt |
| RAG-352 | SEQ ID NO: 105 | UCUAACAGUGCUGUGUCCU[dT][dT] | 21 nt |
| | SEQ ID NO: 106 | AGGACACAGCACUGUUAGA[dT][dT] | 21 nt |
| Hotspot 6 | | | |
| RAG-423 | SEQ ID NO: 107 | UCUUUUCAGCUGCAUUGGG[dT][dT] | 21 nt |
| | SEQ ID NO: 108 | CCCAAUGCAGCUGAAAAGA[dT][dT] | 21 nt |
| RAG-424 | SEQ ID NO: 109 | AUCUUUUCAGCUGCAUUGG[dT][dT] | 21 nt |
| | SEQ ID NO: 110 | CCAAUGCAGCUGAAAAGAU[dT][dT] | 21 nt |
| RAG-425 | SEQ ID NO: 111 | GAUCUUUUCAGCUGCAUUG[dT][dT] | 21 nt |
| | SEQ ID NO: 112 | CAAUGCAGCUGAAAAGAUC[dT][dT] | 21 nt |
| RAG-426 | SEQ ID NO: 113 | UGAUCUUUUCAGCUGCAUU[dT][dT] | 21 nt |
| | SEQ ID NO: 114 | AAUGCAGCUGAAAAGAUCA[dT][dT] | 21 nt |
| RAG-427 | SEQ ID NO: 115 | CUGAUCUUUUCAGCUGCAU[dT][dT] | 21 nt |
| | SEQ ID NO: 116 | AUGCAGCUGAAAAGAUCAG[dT][dT] | 21 nt |
| RAG-428 | SEQ ID NO: 117 | CCUGAUCUUUUCAGCUGCA[dT][dT] | 21 nt |
| | SEQ ID NO: 118 | UGCAGCUGAAAAGAUCAGG[dT][dT] | 21 nt |
| RAG-429 | SEQ ID NO: 119 | UCCUGAUCUUUUCAGCUGC[dT][dT] | 21 nt |
| | SEQ ID NO: 120 | GCAGCUGAAAAGAUCAGGA[dT][dT] | 21 nt |
| RAG-430 | SEQ ID NO: 121 | CUCCUGAUCUUUUCAGCUG[dT][dT] | 21 nt |
| | SEQ ID NO: 122 | CAGCUGAAAAGAUCAGGAG[dT][dT] | 21 nt |
| RAG-431 | SEQ ID NO: 123 | CCUCCUGAUCUUUUCAGCU[dT][dT] | 21 nt |
| | SEQ ID NO: 124 | AGCUGAAAAGAUCAGGAGG[dT][dT] | 21 nt |
| RAG-432 | SEQ ID NO: 125 | UCCUCCUGAUCUUUUCAGC[dT][dT] | 21 nt |
| | SEQ ID NO: 126 | GCUGAAAAGAUCAGGAGGA[dT][dT] | 21 nt |
| RAG-433 | SEQ ID NO: 127 | AUCCUCCUGAUCUUUUCAG[dT][dT] | 21 nt |
| | SEQ ID NO: 128 | CUGAAAAGAUCAGGAGGAU[dT][dT] | 21 nt |
| RAG-434 | SEQ ID NO: 129 | CAUCCUCCUGAUCUUUUCA[dT][dT] | 21 nt |
| | SEQ ID NO: 130 | UGAAAAGAUCAGGAGGAUG[dT][dT] | 21 nt |
| RAG-435 | SEQ ID NO: 131 | UCAUCCUCCUGAUCUUUUC[dT][dT] | 21 nt |
| | SEQ ID NO: 132 | GAAAAGAUCAGGAGGAUGA[dT][dT] | 21 nt |
| RAG-436 | SEQ ID NO: 133 | GUCAUCCUCCUGAUCUUUU[dT][dT] | 21 nt |
| | SEQ ID NO: 134 | AAAAGAUCAGGAGGAUGAC[dT][dT] | 21 nt |
| RAG-437 | SEQ ID NO: 135 | UGUCAUCCUCCUGAUCUUU[dT][dT] | 21 nt |
| | SEQ ID NO: 136 | AAAGAUCAGGAGGAUGACA[dT][dT] | 21 nt |
| RAG-438 | SEQ ID NO: 137 | AUGUCAUCCUCCUGAUCUU[dT][dT] | 21 nt |
| | SEQ ID NO: 138 | AAGAUCAGGAGGAUGACAU[dT][dT] | 21 nt |
| RAG-439 | SEQ ID NO: 139 | AAUGUCAUCCUCCUGAUCU[dT][dT] | 21 nt |
| | SEQ ID NO: 140 | AGAUCAGGAGGAUGACAUU[dT][dT] | 21 nt |
| RAG-440 | SEQ ID NO: 141 | UAAUGUCAUCCUCCUGAUC[dT][dT] | 21 nt |
| | SEQ ID NO: 142 | GAUCAGGAGGAUGACAUUA[dT][dT] | 21 nt |
| RAG-441 | SEQ ID NO: 143 | UUAAUGUCAUCCUCCUGAU[dT][dT] | 21 nt |
| | SEQ ID NO: 144 | AUCAGGAGGAUGACAUUAA[dT][dT] | 21 nt |
| RAG-442 | SEQ ID NO: 145 | AUUAAUGUCAUCCUCCUGA[dT][dT] | 21 nt |
| | SEQ ID NO: 146 | UCAGGAGGAUGACAUUAAU[dT][dT] | 21 nt |
| Hotspot 5 | | | |
| RAG-503 | SEQ ID NO: 147 | UGGGGCUUUUCUGGAAAUU[dT][dT] | 21 nt |
| | SEQ ID NO: 148 | AAUUUCCAGAAAAGCCCCA[dT][dT] | 21 nt |
| RAG-504 | SEQ ID NO: 149 | GUGGGGCUUUUCUGGAAAU[dT][dT] | 21 nt |
| | SEQ ID NO: 150 | AUUUCCAGAAAAGCCCCAC[dT][dT] | 21 nt |
| RAG-505 | SEQ ID NO: 151 | UGUGGGGCUUUUCUGGAAA[dT][dT] | 21 nt |
| | SEQ ID NO: 152 | UUUCCAGAAAAGCCCCACA[dT][dT] | 21 nt |
| RAG-506 | SEQ ID NO: 153 | UUGUGGGGCUUUUCUGGAA[dT][dT] | 21 nt |
| | SEQ ID NO: 154 | UUCCAGAAAAGCCCCACAA[dT][dT] | 21 nt |
| RAG-507 | SEQ ID NO: 155 | AUUGUGGGGCUUUUCUGGA[dT][dT] | 21 nt |
| | SEQ ID NO: 156 | UCCAGAAAAGCCCCACAAU[dT][dT] | 21 nt |
| RAG-508 | SEQ ID NO: 157 | UAUUGUGGGGCUUUUCUGG[dT][dT] | 21 nt |
| | SEQ ID NO: 158 | CCAGAAAAGCCCCACAAUA[dT][dT] | 21 nt |
| RAG-509 | SEQ ID NO: 159 | AUAUUGUGGGGCUUUUCUG[dT][dT] | 21 nt |
| | SEQ ID NO: 160 | CAGAAAAGCCCCACAAUAU[dT][dT] | 21 nt |
| RAG-510 | SEQ ID NO: 161 | GAUAUUGUGGGGCUUUUCU[dT][dT] | 21 nt |
| | SEQ ID NO: 162 | AGAAAAGCCCCACAAUAUC[dT][dT] | 21 nt |
| RAG-511 | SEQ ID NO: 163 | UGAUAUUGUGGGGCUUUUC[dT][dT] | 21 nt |
| | SEQ ID NO: 164 | GAAAAGCCCCACAAUAUCA[dT][dT] | 21 nt |
| RAG-512 | SEQ ID NO: 165 | GUGAUAUUGUGGGGCUUUU[dT][dT] | 21 nt |
| | SEQ ID NO: 166 | AAAAGCCCCACAAUAUCAC[dT][dT] | 21 nt |
| RAG-513 | SEQ ID NO: 167 | GGUGAUAUUGUGGGGCUUU[dT][dT] | 21 nt |
| | SEQ ID NO: 168 | AAAGCCCCACAAUAUCACC[dT][dT] | 21 nt |
| RAG-514 | SEQ ID NO: 169 | AGGUGAUAUUGUGGGGCUU[dT][dT] | 21 nt |
| | SEQ ID NO: 170 | AAGCCCCACAAUAUCACCU[dT][dT] | 21 nt |
| Hotspot 4 | | | |
| RAG-522 | SEQ ID NO: 171 | GGGAAUAGAGGUGAUAUUG[dT][dT] | 21 nt |
| | SEQ ID NO: 172 | CAAUAUCACCUCUAUUCCC[dT][dT] | 21 nt |
| RAG-523 | SEQ ID NO: 173 | UGGGAAUAGAGGUGAUAUU[dT][dT] | 21 nt |
| | SEQ ID NO: 174 | AAUAUCACCUCUAUUCCCA[dT][dT] | 21 nt |
| RAG-524 | SEQ ID NO: 175 | GUGGGAAUAGAGGUGAUAU[dT][dT] | 21 nt |
| | SEQ ID NO: 176 | AUAUCACCUCUAUUCCCAC[dT][dT] | 21 nt |
| RAG-525 | SEQ ID NO: 177 | AGUGGGAAUAGAGGUGAUA[dT][dT] | 21 nt |
| | SEQ ID NO: 178 | UAUCACCUCUAUUCCCACU[dT][dT] | 21 nt |
| RAG-526 | SEQ ID NO: 179 | CAGUGGGAAUAGAGGUGAU[dT][dT] | 21 nt |
| | SEQ ID NO: 180 | AUCACCUCUAUUCCCACUG[dT][dT] | 21 nt |
| RAG-527 | SEQ ID NO: 181 | UCAGUGGGAAUAGAGGUGA[dT][dT] | 21 nt |
| | SEQ ID NO: 182 | UCACCUCUAUUCCCACUGA[dT][dT] | 21 nt |
| RAG-528 | SEQ ID NO: 183 | AUCAGUGGGAAUAGAGGUG[dT][dT] | 21 nt |
| | SEQ ID NO: 184 | CACCUCUAUUCCCACUGAU[dT][dT] | 21 nt |
| RAG-529 | SEQ ID NO: 185 | GAUCAGUGGGAAUAGAGGU[dT][dT] | 21 nt |
| | SEQ ID NO: 186 | ACCUCUAUUCCCACUGAUC[dT][dT] | 21 nt |
| RAG-530 | SEQ ID NO: 187 | GGAUCAGUGGGAAUAGAGG[dT][dT] | 21 nt |
| | SEQ ID NO: 188 | CCUCUAUUCCCACUGAUCC[dT][dT] | 21 nt |
| RAG-531 | SEQ ID NO: 189 | GGGAUCAGUGGGAAUAGAG[dT][dT] | 21 nt |
| | SEQ ID NO: 190 | CUCUAUUCCCACUGAUCCC[dT][dT] | 21 nt |
| RAG-532 | SEQ ID NO: 191 | AGGGAUCAGUGGGAAUAGA[dT][dT] | 21 nt |
| | SEQ ID NO: 192 | UCUAUUCCCACUGAUCCCU[dT][dT] | 21 nt |
| RAG-533 | SEQ ID NO: 193 | GAGGGAUCAGUGGGAAUAG[dT][dT] | 21 nt |
| | SEQ ID NO: 194 | CUAUUCCCACUGAUCCCUC[dT][dT] | 21 nt |
| RAG-534 | SEQ ID NO: 195 | UGAGGGAUCAGUGGGAAUA[dT][dT] | 21 nt |
| | SEQ ID NO: 196 | UAUUCCCACUGAUCCCUCA[dT][dT] | 21 nt |
| RAG-535 | SEQ ID NO: 197 | GUGAGGGAUCAGUGGGAAU[dT][dT] | 21 nt |
| | SEQ ID NO: 198 | AUUCCCACUGAUCCCUCAC[dT][dT] | 21 nt |
| RAG-536 | SEQ ID NO: 199 | AGUGAGGGAUCAGUGGGAA[dT][dT] | 21 nt |
| | SEQ ID NO: 200 | UUCCCACUGAUCCCUCACU[dT][dT] | 21 nt |
| RAG-537 | SEQ ID NO: 201 | UAGUGAGGGAUCAGUGGGA[dT][dT] | 21 nt |
| | SEQ ID NO: 202 | UCCCACUGAUCCCUCACUA[dT][dT] | 21 nt |
| RAG-538 | SEQ ID NO: 203 | CUAGUGAGGGAUCAGUGGG[dT][dT] | 21 nt |
| | SEQ ID NO: 204 | CCCACUGAUCCCUCACUAG[dT][dT] | 21 nt |
| RAG-540 | SEQ ID NO: 205 | ACCUAGUGAGGGAUCAGUG[dT][dT] | 21 nt |
| | SEQ ID NO: 206 | CACUGAUCCCUCACUAGGU[dT][dT] | 21 nt |
| RAG-541 | SEQ ID NO: 207 | GACCUAGUGAGGGAUCAGU[dT][dT] | 21 nt |
| | SEQ ID NO: 208 | ACUGAUCCCUCACUAGGUC[dT][dT] | 21 nt |
| RAG-542 | SEQ ID NO: 209 | UGACCUAGUGAGGGAUCAG[dT][dT] | 21 nt |
| | SEQ ID NO: 210 | CUGAUCCCUCACUAGGUCA[dT][dT] | 21 nt |
| RAG-543 | SEQ ID NO: 211 | GUGACCUAGUGAGGGAUCA[dT][dT] | 21 nt |
| | SEQ ID NO: 212 | UGAUCCCUCACUAGGUCAC[dT][dT] | 21 nt |
| RAG-544 | SEQ ID NO: 213 | GGUGACCUAGUGAGGGAUC[dT][dT] | 21 nt |
| | SEQ ID NO: 214 | GAUCCCUCACUAGGUCACC[dT][dT] | 21 nt |
| RAG-545 | SEQ ID NO: 215 | AGGUGACCUAGUGAGGGAU[dT][dT] | 21 nt |
| | SEQ ID NO: 216 | AUCCCUCACUAGGUCACCU[dT][dT] | 21 nt |
| RAG-546 | SEQ ID NO: 217 | GAGGUGACCUAGUGAGGGA[dT][dT] | 21 nt |
| | SEQ ID NO: 218 | UCCCUCACUAGGUCACCUC[dT][dT] | 21 nt |
| RAG-549 | SEQ ID NO: 219 | GGAGAGGUGACCUAGUGAG[dT][dT] | 21 nt |
| | SEQ ID NO: 220 | CUCACUAGGUCACCUCUCC[dT][dT] | 21 nt |
| RAG-550 | SEQ ID NO: 221 | GGGAGAGGUGACCUAGUGA[dT][dT] | 21 nt |
| | SEQ ID NO: 222 | UCACUAGGUCACCUCUCCC[dT][dT] | 21 nt |
| RAG-551 | SEQ ID NO: 223 | UGGGAGAGGUGACCUAGUG[dT][dT] | 21 nt |
| | SEQ ID NO: 224 | CACUAGGUCACCUCUCCCA[dT][dT] | 21 nt |
| RAG-552 | SEQ ID NO: 225 | CUGGGAGAGGUGACCUAGU[dT][dT] | 21 nt |
| | SEQ ID NO: 226 | ACUAGGUCACCUCUCCCAG[dT][dT] | 21 nt |
| RAG-553 | SEQ ID NO: 227 | UCUGGGAGAGGUGACCUAG[dT][dT] | 21 nt |
| | SEQ ID NO: 228 | CUAGGUCACCUCUCCCAGA[dT][dT] | 21 nt |
| RAG-554 | SEQ ID NO: 229 | UUCUGGGAGAGGUGACCUA[dT][dT] | 21 nt |
| | SEQ ID NO: 230 | UAGGUCACCUCUCCCAGAA[dT][dT] | 21 nt |
| Hotspot 3 | | | |
| RAG-569 | SEQ ID NO: 231 | AGGUGCUCCAGGUGCUUCU[dT][dT] | 21 nt |
| | SEQ ID NO: 232 | AGAAGCACCUGGAGCACCU[dT][dT] | 21 nt |
| RAG-570 | SEQ ID NO: 233 | UAGGUGCUCCAGGUGCUUC[dT][dT] | 21 nt |
| | SEQ ID NO: 234 | GAAGCACCUGGAGCACCUA[dT][dT] | 21 nt |
| RAG-574 | SEQ ID NO: 235 | UGUCUAGGUGCUCCAGGUG[dT][dT] | 21 nt |
| | SEQ ID NO: 236 | CACCUGGAGCACCUAGACA[dT][dT] | 21 nt |
| RAG-576 | SEQ ID NO: 237 | GGUGUCUAGGUGCUCCAGG[dT][dT] | 21 nt |
| | SEQ ID NO: 238 | CCUGGAGCACCUAGACACC[dT][dT] | 21 nt |
| RAG-577 | SEQ ID NO: 239 | GGGUGUCUAGGUGCUCCAG[dT][dT] | 21 nt |
| | SEQ ID NO: 240 | CUGGAGCACCUAGACACCC[dT][dT] | 21 nt |
| RAG-578 | SEQ ID NO: 241 | GGGGUGUCUAGGUGCUCCA[dT][dT] | 21 nt |
| | SEQ ID NO: 242 | UGGAGCACCUAGACACCCC[dT][dT] | 21 nt |
| RAG-579 | SEQ ID NO: 243 | UGGGGUGUCUAGGUGCUCC[dT][dT] | 21 nt |
| | SEQ ID NO: 244 | GGAGCACCUAGACACCCCA[dT][dT] | 21 nt |
| RAG-580 | SEQ ID NO: 245 | UUGGGGUGUCUAGGUGCUC[dT][dT] | 21 nt |
| | SEQ ID NO: 246 | GAGCACCUAGACACCCCAA[dT][dT] | 21 nt |
| RAG-583 | SEQ ID NO: 247 | UUGUUGGGGUGUCUAGGUG[dT][dT] | 21 nt |
| | SEQ ID NO: 248 | CACCUAGACACCCCAACAA[dT][dT] | 21 nt |
| RAG-584 | SEQ ID NO: 249 | UUUGUUGGGGUGUCUAGGU[dT][dT] | 21 nt |
| | SEQ ID NO: 250 | ACCUAGACACCCCAACAAA[dT][dT] | 21 nt |
| RAG-585 | SEQ ID NO: 251 | CUUUGUUGGGGUGUCUAGG[dT][dT] | 21 nt |
| | SEQ ID NO: 252 | CCUAGACACCCCAACAAAG[dT][dT] | 21 nt |
| Hotspot 2 | | | |
| RAG-650 | SEQ ID NO: 253 | AGUGGACCUCAAUUUCCUC[dT][dT] | 21 nt |
| | SEQ ID NO: 254 | GAGGAAAUUGAGGUCCACU[dT][dT] | 21 nt |
| RAG-651 | SEQ ID NO: 255 | CAGUGGACCUCAAUUUCCU[dT][dT] | 21 nt |
| | SEQ ID NO: 256 | AGGAAAUUGAGGUCCACUG[dT][dT] | 21 nt |
| RAG-652 | SEQ ID NO: 257 | UCAGUGGACCUCAAUUUCC[dT][dT] | 21 nt |
| | SEQ ID NO: 258 | GGAAAUUGAGGUCCACUGA[dT][dT] | 21 nt |
| RAG-653 | SEQ ID NO: 259 | UUCAGUGGACCUCAAUUUC[dT][dT] | 21 nt |
| | SEQ ID NO: 260 | GAAAUUGAGGUCCACUGAA[dT][dT] | 21 nt |
| RAG-654 | SEQ ID NO: 261 | GUUCAGUGGACCUCAAUUU[dT][dT] | 21 nt |
| | SEQ ID NO: 262 | AAAUUGAGGUCCACUGAAC[dT][dT] | 21 nt |
| RAG-655 | SEQ ID NO: 263 | AGUUCAGUGGACCUCAAUU[dT][dT] | 21 nt |
| | SEQ ID NO: 264 | AAUUGAGGUCCACUGAACU[dT][dT] | 21 nt |
| RAG-656 | SEQ ID NO: 265 | AAGUUCAGUGGACCUCAAU[dT][dT] | 21 nt |
| | SEQ ID NO: 266 | AUUGAGGUCCACUGAACUU[dT][dT] | 21 nt |
| RAG-657 | SEQ ID NO: 267 | UAAGUUCAGUGGACCUCAA[dT][dT] | 21 nt |
| | SEQ ID NO: 268 | UUGAGGUCCACUGAACUUA[dT][dT] | 21 nt |
| RAG-658 | SEQ ID NO: 269 | UUAAGUUCAGUGGACCUCA[dT][dT] | 21 nt |
| | SEQ ID NO: 270 | UGAGGUCCACUGAACUUAA[dT][dT] | 21 nt |
| RAG-659 | SEQ ID NO: 271 | CUUAAGUUCAGUGGACCUC[dT][dT] | 21 nt |
| | SEQ ID NO: 272 | GAGGUCCACUGAACUUAAG[dT][dT] | 21 nt |
| RAG-660 | SEQ ID NO: 273 | ACUUAAGUUCAGUGGACCU[dT][dT] | 21 nt |
| | SEQ ID NO: 274 | AGGUCCACUGAACUUAAGU[dT][dT] | 21 nt |
| RAG-661 | SEQ ID NO: 275 | UACUUAAGUUCAGUGGACC[dT][dT] | 21 nt |
| | SEQ ID NO: 276 | GGUCCACUGAACUUAAGUA[dT][dT] | 21 nt |
| RAG-662 | SEQ ID NO: 277 | AUACUUAAGUUCAGUGGAC[dT][dT] | 21 nt |
| | SEQ ID NO: 278 | GUCCACUGAACUUAAGUAU[dT][dT] | 21 nt |
| RAG-682 | SEQ ID NO: 279 | GUGACAAUCAACAACUUUG[dT][dT] | 21 nt |
| | SEQ ID NO: 280 | CAAAGUUGUUGAUUGUCAC[dT][dT] | 21 nt |
| RAG-685 | SEQ ID NO: 281 | CAUGUGACAAUCAACAACU[dT][dT] | 21 nt |
| | SEQ ID NO: 282 | AGUUGUUGAUUGUCACAUG[dT][dT] | 21 nt |
| RAG-686 | SEQ ID NO: 283 | GCAUGUGACAAUCAACAAC[dT][dT] | 21 nt |
| | SEQ ID NO: 284 | GUUGUUGAUUGUCACAUGC[dT][dT] | 21 nt |
| RAG-687 | SEQ ID NO: 285 | AGCAUGUGACAAUCAACAA[dT][dT] | 21 nt |
| | SEQ ID NO: 286 | UUGUUGAUUGUCACAUGCU[dT][dT] | 21 nt |
| RAG-688 | SEQ ID NO: 287 | AAGCAUGUGACAAUCAACA[dT][dT] | 21 nt |
| | SEQ ID NO: 288 | UGUUGAUUGUCACAUGCUU[dT][dT] | 21 nt |
| RAG-689 | SEQ ID NO: 289 | GAAGCAUGUGACAAUCAAC[dT][dT] | 21 nt |
| | SEQ ID NO: 290 | GUUGAUUGUCACAUGCUUC[dT][dT] | 21 nt |
| RAG-690 | SEQ ID NO: 291 | GGAAGCAUGUGACAAUCAA[dT][dT] | 21 nt |
| | SEQ ID NO: 292 | UUGAUUGUCACAUGCUUCC[dT][dT] | 21 nt |
| RAG-691 | SEQ ID NO: 293 | CGGAAGCAUGUGACAAUCA[dT][dT] | 21 nt |
| | SEQ ID NO: 294 | UGAUUGUCACAUGCUUCCG[dT][dT] | 21 nt |
| RAG-692 | SEQ ID NO: 295 | CCGGAAGCAUGUGACAAUC[dT][dT] | 21 nt |
| | SEQ ID NO: 296 | GAUUGUCACAUGCUUCCGG[dT][dT] | 21 nt |
| RAG-693 | SEQ ID NO: 297 | CCCGGAAGCAUGUGACAAU[dT][dT] | 21 nt |
| | SEQ ID NO: 298 | AUUGUCACAUGCUUCCGGG[dT][dT] | 21 nt |
| RAG-694 | SEQ ID NO: 299 | UCCCGGAAGCAUGUGACAA[dT][dT] | 21 nt |
| | SEQ ID NO: 300 | UUGUCACAUGCUUCCGGGA[dT][dT] | 21 nt |
| RAG-695 | SEQ ID NO: 301 | UUCCCGGAAGCAUGUGACA[dT][dT] | 21 nt |
| | SEQ ID NO: 302 | UGUCACAUGCUUCCGGGAA[dT][dT] | 21 nt |
| RAG-696 | SEQ ID NO: 303 | CUUCCCGGAAGCAUGUGAC[dT][dT] | 21 nt |
| | SEQ ID NO: 304 | GUCACAUGCUUCCGGGAAG[dT][dT] | 21 nt |
| RAG-697 | SEQ ID NO: 305 | CCUUCCCGGAAGCAUGUGA[dT][dT] | 21 nt |
| | SEQ ID NO: 306 | UCACAUGCUUCCGGGAAGG[dT][dT] | 21 nt |
| RAG-698 | SEQ ID NO: 307 | UCCUUCCCGGAAGCAUGUG[dT][dT] | 21 nt |
| | SEQ ID NO: 308 | CACAUGCUUCCGGGAAGGA[dT][dT] | 21 nt |
| RAG-699 | SEQ ID NO: 309 | CUCCUUCCCGGAAGCAUGU[dT][dT] | 21 nt |
| | SEQ ID NO: 310 | ACAUGCUUCCGGGAAGGAG[dT][dT] | 21 nt |
| RAG-700 | SEQ ID NO: 311 | CCUCCUUCCCGGAAGCAUG[dT][dT] | 21 nt |
| | SEQ ID NO: 312 | CAUGCUUCCGGGAAGGAGG[dT][dT] | 21 nt |
| RAG-701 | SEQ ID NO: 313 | CCCUCCUUCCCGGAAGCAU[dT][dT] | 21 nt |
| | SEQ ID NO: 314 | AUGCUUCCGGGAAGGAGGG[dT][dT] | 21 nt |
| RAG-702 | SEQ ID NO: 315 | UCCCUCCUUCCCGGAAGCA[dT][dT] | 21 nt |
| | SEQ ID NO: 316 | UGCUUCCGGGAAGGAGGGA[dT][dT] | 21 nt |
| RAG-704 | SEQ ID NO: 317 | AUUCCCUCCUUCCCGGAAG[dT][dT] | 21 nt |
| | SEQ ID NO: 318 | CUUCCGGGAAGGAGGGAAU[dT][dT] | 21 nt |
| RAG-705 | SEQ ID NO: 319 | AAUUCCCUCCUUCCCGGAA[dT][dT] | 21 nt |
| | SEQ ID NO: 320 | UUCCGGGAAGGAGGGAAUU[dT][dT] | 21 nt |
| RAG-710 | SEQ ID NO: 321 | UCUCCAAUUCCCUCCUUCC[dT][dT] | 21 nt |
| | SEQ ID NO: 322 | GGAAGGAGGGAAUUGGAGA[dT][dT] | 21 nt |
| RAG-711 | SEQ ID NO: 323 | CUCUCCAAUUCCCUCCUUC[dT][dT] | 21 nt |
| | SEQ ID NO: 324 | GAAGGAGGGAAUUGGAGAG[dT][dT] | 21 nt |
| RAG-712 | SEQ ID NO: 325 | UCUCUCCAAUUCCCUCCUU[dT][dT] | 21 nt |
| | SEQ ID NO: 326 | AAGGAGGGAAUUGGAGAGA[dT][dT] | 21 nt |
| RAG-713 | SEQ ID NO: 327 | GUCUCUCCAAUUCCCUCCU[dT][dT] | 21 nt |
| | SEQ ID NO: 328 | AGGAGGGAAUUGGAGAGAC[dT][dT] | 21 nt |
| RAG-714 | SEQ ID NO: 329 | AGUCUCUCCAAUUCCCUCC[dT][dT] | 21 nt |
| | SEQ ID NO: 330 | GGAGGGAAUUGGAGAGACU[dT][dT] | 21 nt |
| RAG-715 | SEQ ID NO: 331 | UAGUCUCUCCAAUUCCCUC[dT][dT] | 21 nt |
| | SEQ ID NO: 332 | GAGGGAAUUGGAGAGACUA[dT][dT] | 21 nt |
| RAG-716 | SEQ ID NO: 333 | GUAGUCUCUCCAAUUCCCU[dT][dT] | 21 nt |
| | SEQ ID NO: 334 | AGGGAAUUGGAGAGACUAC[dT][dT] | 21 nt |
| RAG-717 | SEQ ID NO: 335 | GGUAGUCUCUCCAAUUCCC[dT][dT] | 21 nt |
| | SEQ ID NO: 336 | GGGAAUUGGAGAGACUACC[dT][dT] | 21 nt |
| Hotspot 1 | | | |
| RAG-820 | SEQ ID NO: 337 | UGCAACCACAGGGAUUUCU[dT][dT] | 21 nt |
| | SEQ ID NO: 338 | AGAAAUCCCUGUGGUUGCA[dT][dT] | 21 nt |
| RAG-821 | SEQ ID NO: 339 | CUGCAACCACAGGGAUUUC[dT][dT] | 21 nt |
| | SEQ ID NO: 340 | GAAAUCCCUGUGGUUGCAG[dT][dT] | 21 nt |
| RAG-822 | SEQ ID NO: 341 | GCUGCAACCACAGGGAUUU[dT][dT] | 21 nt |
| | SEQ ID NO: 342 | AAAUCCCUGUGGUUGCAGC[dT][dT] | 21 nt |
| RAG-823 | SEQ ID NO: 343 | UGCUGCAACCACAGGGAUU[dT][dT] | 21 nt |
| | SEQ ID NO: 344 | AAUCCCUGUGGUUGCAGCA[dT][dT] | 21 nt |
| RAG-824 | SEQ ID NO: 345 | CUGCUGCAACCACAGGGAU[dT][dT] | 21 nt |
| | SEQ ID NO: 346 | AUCCCUGUGGUUGCAGCAG[dT][dT] | 21 nt |
| RAG-825 | SEQ ID NO: 347 | GCUGCUGCAACCACAGGGA[dT][dT] | 21 nt |
| | SEQ ID NO: 348 | UCCCUGUGGUUGCAGCAGC[dT][dT] | 21 nt |
| RAG-826 | SEQ ID NO: 349 | AGCUGCUGCAACCACAGGG[dT][dT] | 21 nt |
| | SEQ ID NO: 350 | CCCUGUGGUUGCAGCAGCU[dT][dT] | 21 nt |
| RAG-828 | SEQ ID NO: 351 | AAAGCUGCUGCAACCACAG[dT][dT] | 21 nt |
| | SEQ ID NO: 352 | CUGUGGUUGCAGCAGCUUU[dT][dT] | 21 nt |
| RAG-829 | SEQ ID NO: 353 | CAAAGCUGCUGCAACCACA[dT][dT] | 21 nt |
| | SEQ ID NO: 354 | UGUGGUUGCAGCAGCUUUG[dT][dT] | 21 nt |
| RAG-830 | SEQ ID NO: 355 | ACAAAGCUGCUGCAACCAC[dT][dT] | 21 nt |
| | SEQ ID NO: 356 | GUGGUUGCAGCAGCUUUGU[dT][dT] | 21 nt |
| RAG-831 | SEQ ID NO: 357 | AACAAAGCUGCUGCAACCA[dT][dT] | 21 nt |
| | SEQ ID NO: 358 | UGGUUGCAGCAGCUUUGUU[dT][dT] | 21 nt |
| RAG-832 | SEQ ID NO: 359 | CAACAAAGCUGCUGCAACC[dT][dT] | 21 nt |
| | SEQ ID NO: 360 | GGUUGCAGCAGCUUUGUUG[dT][dT] | 21 nt |
| RAG-833 | SEQ ID NO: 361 | CCAACAAAGCUGCUGCAAC[dT][dT] | 21 nt |
| | SEQ ID NO: 362 | GUUGCAGCAGCUUUGUUGG[dT][dT] | 21 nt |
| RAG-834 | SEQ ID NO: 363 | GCCAACAAAGCUGCUGCAA[dT][dT] | 21 nt |
| | SEQ ID NO: 364 | UUGCAGCAGCUUUGUUGGC[dT][dT] | 21 nt |
| RAG-835 | SEQ ID NO: 365 | GGCCAACAAAGCUGCUGCA[dT][dT] | 21 nt |
| | SEQ ID NO: 366 | UGCAGCAGCUUUGUUGGCC[dT][dT] | 21 nt |
| RAG-836 | SEQ ID NO: 367 | UGGCCAACAAAGCUGCUGC[dT][dT] | 21 nt |
| | SEQ ID NO: 368 | GCAGCAGCUUUGUUGGCCA[dT][dT] | 21 nt |
| RAG-837 | SEQ ID NO: 369 | CUGGCCAACAAAGCUGCUG[dT][dT] | 21 nt |
| | SEQ ID NO: 370 | CAGCAGCUUUGUUGGCCAG[dT][dT] | 21 nt |
| RAG-838 | SEQ ID NO: 371 | CCUGGCCAACAAAGCUGCU[dT][dT] | 21 nt |
| | SEQ ID NO: 372 | AGCAGCUUUGUUGGCCAGG[dT][dT] | 21 nt |
| RAG-840 | SEQ ID NO: 373 | UUCCUGGCCAACAAAGCUG[dT][dT] | 21 nt |
| | SEQ ID NO: 374 | CAGCUUUGUUGGCCAGGAA[dT][dT] | 21 nt |
| RAG-841 | SEQ ID NO: 375 | CUUCCUGGCCAACAAAGCU[dT][dT] | 21 nt |
| | SEQ ID NO: 376 | AGCUUUGUUGGCCAGGAAG[dT][dT] | 21 nt |
| RAG-843 | SEQ ID NO: 377 | CCCUUCCUGGCCAACAAAG[dT][dT] | 21 nt |
| | SEQ ID NO: 378 | CUUUGUUGGCCAGGAAGGG[dT][dT] | 21 nt |
| RAG-844 | SEQ ID NO: 379 | CCCCUUCCUGGCCAACAAA[dT][dT] | 21 nt |
| | SEQ ID NO: 380 | UUUGUUGGCCAGGAAGGGG[dT][dT] | 21 nt |
| RAG-845 | SEQ ID NO: 381 | UCCCCUUCCUGGCCAACAA[dT][dT] | 21 nt |
| | SEQ ID NO: 382 | UUGUUGGCCAGGAAGGGGA[dT][dT] | 21 nt |
| RAG-846 | SEQ ID NO: 383 | CUCCCCUUCCUGGCCAACA[dT][dT] | 21 nt |
| | SEQ ID NO: 384 | UGUUGGCCAGGAAGGGGAG[dT][dT] | 21 nt |
| RAG-848 | SEQ ID NO: 385 | UCCUCCCCUUCCUGGCCAA[dT][dT] | 21 nt |
| | SEQ ID NO: 386 | UUGGCCAGGAAGGGGAGGA[dT][dT] | 21 nt |
| RAG-849 | SEQ ID NO: 387 | AUCCUCCCCUUCCUGGCCA[dT][dT] | 21 nt |
| | SEQ ID NO: 388 | UGGCCAGGAAGGGGAGGAU[dT][dT] | 21 nt |
| RAG-853 | SEQ ID NO: 389 | UCAAAUCCUCCCCUUCCUG[dT][dT] | 21 nt |
| | SEQ ID NO: 390 | CAGGAAGGGGAGGAUUUGA[dT][dT] | 21 nt |
| RAG-854 | SEQ ID NO: 391 | GUCAAAUCCUCCCCUUCCU[dT][dT] | 21 nt |
| | SEQ ID NO: 392 | AGGAAGGGGAGGAUUUGAC[dT][dT] | 21 nt |
| RAG-855 | SEQ ID NO: 393 | CGUCAAAUCCUCCCCUUCC[dT][dT] | 21 nt |
| | SEQ ID NO: 394 | GGAAGGGGAGGAUUUGACG[dT][dT] | 21 nt |
| RAG-856 | SEQ ID NO: 395 | UCGUCAAAUCCUCCCCUUC[dT][dT] | 21 nt |
| | SEQ ID NO: 396 | GAAGGGGAGGAUUUGACGA[dT][dT] | 21 nt |
| RAG-857 | SEQ ID NO: 397 | CUCGUCAAAUCCUCCCCUU[dT][dT] | 21 nt |
| | SEQ ID NO: 398 | AAGGGGAGGAUUUGACGAG[dT][dT] | 21 nt |
| RAG-858 | SEQ ID NO: 399 | ACUCGUCAAAUCCUCCCCU[dT][dT] | 21 nt |
| | SEQ ID NO: 400 | AGGGGAGGAUUUGACGAGU[dT][dT] | 21 nt |
| RAG-860 | SEQ ID NO: 401 | UCACUCGUCAAAUCCUCCC[dT][dT] | 21 nt |
| | SEQ ID NO: 402 | GGGAGGAUUUGACGAGUGA[dT][dT] | 21 nt |
| RAG-861 | SEQ ID NO: 403 | CUCACUCGUCAAAUCCUCC[dT][dT] | 21 nt |
| | SEQ ID NO: 404 | GGAGGAUUUGACGAGUGAG[dT][dT] | 21 nt |
| RAG-862 | SEQ ID NO: 405 | ACUCACUCGUCAAAUCCUC[dT][dT] | 21 nt |
| | SEQ ID NO: 406 | GAGGAUUUGACGAGUGAGU[dT][dT] | 21 nt |
| RAG-864 | SEQ ID NO: 407 | CAACUCACUCGUCAAAUCC[dT][dT] | 21 nt |
| | SEQ ID NO: 408 | GGAUUUGACGAGUGAGUUG[dT][dT] | 21 nt |
| RAG-865 | SEQ ID NO: 409 | ACAACUCACUCGUCAAAUC[dT][dT] | 21 nt |
| | SEQ ID NO: 410 | GAUUUGACGAGUGAGUUGU[dT][dT] | 21 nt |
| RAG-866 | SEQ ID NO: 411 | GACAACUCACUCGUCAAAU[dT][dT] | 21 nt |
| | SEQ ID NO: 412 | AUUUGACGAGUGAGUUGUC[dT][dT] | 21 nt |
| RAG-867 | SEQ ID NO: 413 | AGACAACUCACUCGUCAAA[dT][dT] | 21 nt |
| | SEQ ID NO: 414 | UUUGACGAGUGAGUUGUCU[dT][dT] | 21 nt |
| RAG-868 | SEQ ID NO: 415 | CAGACAACUCACUCGUCAA[dT][dT] | 21 nt |
| | SEQ ID NO: 416 | UUGACGAGUGAGUUGUCUG[dT][dT] | 21 nt |
| RAG-869 | SEQ ID NO: 417 | ACAGACAACUCACUCGUCA[dT][dT] | 21 nt |
| | SEQ ID NO: 418 | UGACGAGUGAGUUGUCUGU[dT][dT] | 21 nt |
| RAG-870 | SEQ ID NO: 419 | GACAGACAACUCACUCGUC[dT][dT] | 21 nt |
| | SEQ ID NO: 420 | GACGAGUGAGUUGUCUGUC[dT][dT] | 21 nt |
| RAG-871 | SEQ ID NO: 421 | AGACAGACAACUCACUCGU[dT][dT] | 21 nt |
| | SEQ ID NO: 422 | ACGAGUGAGUUGUCUGUCU[dT][dT] | 21 nt |
| RAG-872 | SEQ ID NO: 423 | GAGACAGACAACUCACUCG[dT][dT] | 21 nt |
| | SEQ ID NO: 424 | CGAGUGAGUUGUCUGUCUC[dT][dT] | 21 nt |
| RAG-873 | SEQ ID NO: 425 | GGAGACAGACAACUCACUC[dT][dT] | 21 nt |
| | SEQ ID NO: 426 | GAGUGAGUUGUCUGUCUCC[dT][dT] | 21 nt |
| RAG-874 | SEQ ID NO: 427 | AGGAGACAGACAACUCACU[dT][dT] | 21 nt |
| | SEQ ID NO: 428 | AGUGAGUUGUCUGUCUCCU[dT][dT] | 21 nt |
| RAG-875 | SEQ ID NO: 429 | CAGGAGACAGACAACUCAC[dT][dT] | 21 nt |
| | SEQ ID NO: 430 | GUGAGUUGUCUGUCUCCUG[dT][dT] | 21 nt |
| RAG-876 | SEQ ID NO: 431 | UCAGGAGACAGACAACUCA[dT][dT] | 21 nt |
| | SEQ ID NO: 432 | UGAGUUGUCUGUCUCCUGA[dT][dT] | 21 nt |
| RAG-877 | SEQ ID NO: 433 | UUCAGGAGACAGACAACUC[dT][dT] | 21 nt |
| | SEQ ID NO: 434 | GAGUUGUCUGUCUCCUGAA[dT][dT] | 21 nt |
| RAG-878 | SEQ ID NO: 435 | AUUCAGGAGACAGACAACU[dT][dT] | 21 nt |
| | SEQ ID NO: 436 | AGUUGUCUGUCUCCUGAAU[dT][dT] | 21 nt |
| RAG-879 | SEQ ID NO: 437 | UAUUCAGGAGACAGACAAC[dT][dT] | 21 nt |
| | SEQ ID NO: 438 | GUUGUCUGUCUCCUGAAUA[dT][dT] | 21 nt |
| RAG-880 | SEQ ID NO: 439 | GUAUUCAGGAGACAGACAA[dT][dT] | 21 nt |
| | SEQ ID NO: 440 | UUGUCUGUCUCCUGAAUAC[dT][dT] | 21 nt |
| RAG-881 | SEQ ID NO: 441 | AGUAUUCAGGAGACAGACA[dT][dT] | 21 nt |
| | SEQ ID NO: 442 | UGUCUGUCUCCUGAAUACU[dT][dT] | 21 nt |
| RAG-882 | SEQ ID NO: 443 | GAGUAUUCAGGAGACAGAC[dT][dT] | 21 nt |
| | SEQ ID NO: 444 | GUCUGUCUCCUGAAUACUC[dT][dT] | 21 nt |
| RAG-883 | SEQ ID NO: 445 | GGAGUAUUCAGGAGACAGA[dT][dT] | 21 nt |
| | SEQ ID NO: 446 | UCUGUCUCCUGAAUACUCC[dT][dT] | 21 nt |
| RAG-884 | SEQ ID NO: 447 | GGGAGUAUUCAGGAGACAG[dT][dT] | 21 nt |
| | SEQ ID NO: 448 | CUGUCUCCUGAAUACUCCC[dT][dT] | 21 nt |
| RAG-885 | SEQ ID NO: 449 | GGGGAGUAUUCAGGAGACA[dT][dT] | 21 nt |
| | SEQ ID NO: 450 | UGUCUCCUGAAUACUCCCC[dT][dT] | 21 nt |
| RAG-886 | SEQ ID NO: 451 | UGGGGAGUAUUCAGGAGAC[dT][dT] | 21 nt |
| | SEQ ID NO: 452 | GUCUCCUGAAUACUCCCCA[dT][dT] | 21 nt |
| RAG-887 | SEQ ID NO: 453 | GUGGGGAGUAUUCAGGAGA[dT][dT] | 21 nt |
| | SEQ ID NO: 454 | UCUCCUGAAUACUCCCCAC[dT][dT] | 21 nt |
| RAG-888 | SEQ ID NO: 455 | UGUGGGGAGUAUUCAGGAG[dT][dT] | 21 nt |
| | SEQ ID NO: 456 | CUCCUGAAUACUCCCCACA[dT][dT] | 21 nt |
| RAG-889 | SEQ ID NO: 457 | AUGUGGGGAGUAUUCAGGA[dT][dT] | 21 nt |
| | SEQ ID NO: 458 | UCCUGAAUACUCCCCACAU[dT][dT] | 21 nt |
| RAG-890 | SEQ ID NO: 459 | UAUGUGGGGAGUAUUCAGG[dT][dT] | 21 nt |
| | SEQ ID NO: 460 | CCUGAAUACUCCCCACAUA[dT][dT] | 21 nt |
| RAG-891 | SEQ ID NO: 461 | CUAUGUGGGGAGUAUUCAG[dT][dT] | 21 nt |
| | SEQ ID NO: 462 | CUGAAUACUCCCCACAUAG[dT][dT] | 21 nt |
| RAG-892 | SEQ ID NO: 463 | GCUAUGUGGGGAGUAUUCA[dT][dT] | 21 nt |
| | SEQ ID NO: 464 | UGAAUACUCCCCACAUAGC[dT][dT] | 21 nt |
| RAG-893 | SEQ ID NO: 465 | GGCUAUGUGGGGAGUAUUC[dT][dT] | 21 nt |
| | SEQ ID NO: 466 | GAAUACUCCCCACAUAGCC[dT][dT] | 21 nt |
| RAG-894 | SEQ ID NO: 467 | GGGCUAUGUGGGGAGUAUU[dT][dT] | 21 nt |
| | SEQ ID NO: 468 | AAUACUCCCCACAUAGCCC[dT][dT] | 21 nt |

These hotspots include: hotspot 1 having a corresponding target sequence from −893 bp to −801 bp in the p21 promoter sequence, shown as SEQ ID NO: 93, wherein 44 functional saRNAs (Table 3, FIG. 3A) were discovered in this region, comprising RAG-834, RAG-845, RAG-892, RAG-846, RAG-821, RAG-884, RAG-864, RAG-843, RAG-854, RAG-844, RAG-887, RAG-838, RAG-858, RAG-835, RAG-876, RAG-870, RAG-853, RAG-881, RAG-828, RAG-872, RAG-841, RAG-831, RAG-829, RAG-820, RAG-822, RAG-868, RAG-849, RAG-862, RAG-865, RAG-893, RAG-848, RAG-824, RAG-866, RAG-840, RAG-875, RAG-880, RAG-871, RAG-888, RAG-885, RAG-894, RAG-833, RAG-825, RAG-889, and RAG-823;

hotspot 2 (Table 3, FIG. 3B) having a corresponding target sequence from −717 bp to −632 bp in the p21 promoter sequence, shown as SEQ ID NO: 94, wherein 31 functional saRNAs were discovered in this region, comprising RAG-693, RAG-692, RAG-688, RAG-696, RAG-694, RAG-687, RAG-691, RAG-690, RAG-689, RAG-682, RAG-686, RAG-662, RAG-695, RAG-654, RAG-658, RAG-685, RAG-704, RAG-714, RAG-705, RAG-661, RAG-656, RAG-698, RAG-697, RAG-657, RAG-715, RAG-652, RAG-651, RAG-650, RAG-716, RAG-717, and RAG-711;

hotspot 3 (Table 3, FIG. 3C) having a corresponding target sequence from −585 bp to −551 bp in the p21 promoter sequence, shown as SEQ ID NO: 95, wherein 9 functional saRNAs were discovered in this region, comprising RAG-580, RAG-577, RAG-569, RAG-576, RAG-570, RAG-574, RAG-585, RAG-579, and RAG-584;

hotspot 4 (Table 3, FIG. 3D) having a corresponding target sequence from −554 bp to −505 bp in the p21 promoter sequence, shown as SEQ ID NO: 96, wherein 17 functional saRNAs were discovered in this region, comprising RAG-524, RAG-553, RAG-537, RAG-526, RAG-554, RAG-523, RAG-534, RAG-543, RAG-525, RAG-535, RAG-546, RAG-545, RAG-542, RAG-531, RAG-522, RAG-529, and RAG-552;

hotspot 5 (Table 3, FIG. 3E) having a corresponding target sequence from −514 bp to −485 bp in the p21 promoter sequence, shown as SEQ ID NO: 97, wherein 9 functional saRNAs were discovered in this region, comprising RAG-503, RAG-504, RAG-505, RAG-506, RAG-507, RAG-508, RAG-509, RAG-510, RAG-511, RAG-512, RAG-513, and RAG-514;

hotspot 6 (Table 3, FIG. 3F) having a corresponding target sequence from −442 bp to −405 bp in the p21 promoter sequence, shown as SEQ ID NO: 98, wherein 12 functional saRNAs were discovered in this region, comprising RAG-427, RAG-430, RAG-431, RAG-423, RAG-425, RAG-433, RAG-435, RAG-434, RAG-439, RAG-426, RAG-428, and RAG-442;

hotspot 7 (Table 3, FIG. 3G) having a corresponding target sequence from −352 bp to −313 bp in the p21 promoter sequence, shown as SEQ ID NO: 99, wherein 13 functional saRNAs were discovered in this region, comprising RAG-335, RAG-351, RAG-352, RAG-331, RAG-344, RAG-342, RAG-341, RAG-333, RAG-345, RAG-346, RAG-336, RAG-332, and RAG-343;

and hotspot 8 (Table 3, FIG. 3H) having a corresponding target sequence from −325 bp to −260 bp in the p21 promoter sequence, shown as SEQ ID NO: 100, wherein 18 functional saRNAs were discovered in this region, comprising RAG-294, RAG-285, RAG-286, RAG-292, RAG-291, RAG-284, RAG-279, RAG-280, RAG-325, RAG-293, RAG-322, RAG-321, RAG-281, RAG-289, RAG-278, RAG-283, RAG-282, and RAG-295.

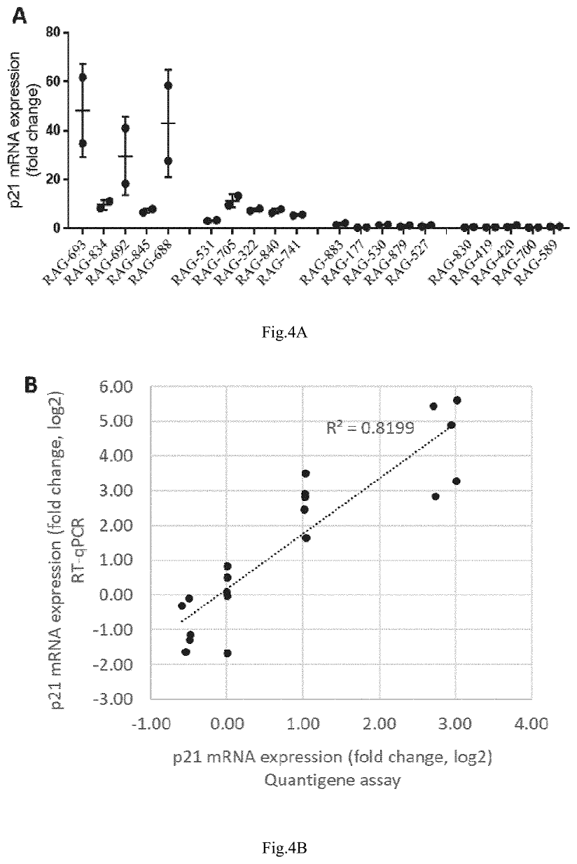

In order to verify the QuantiGene 2.0 assay results, the 439 double-stranded RNA molecules were divided into four bins according to their activities in inducing p21 mRNA expression, and 5 double-stranded RNA molecules were randomly selected from each bin and transfected into PC3 cells at a concentration of 10 nM. 72 hours after transfection, total cellular RNA was extracted from the transfected cells and reverse transcribed into cDNA which was amplified by RT-qPCR to determine p21 mRNA level. p21 mRNA expression levels for cells transfected with each of the saRNAs determined by the two methods showed a significant correlation (R²=0.82) (FIG. 4). All selected functional saRNAs obtained through the QuantiGene 2.0 method were verified as real functional saRNAs by RT-qPCR, and some of them exhibited even a stronger p21 mRNA induction ability by the RT-qPCR (Table 4).

TABLE 4
Verification for QuantiGene 2.0 method

Relative p21 mRNA level

| Bin | Title | Quantigene 2.0 | RT-qPCR |
|---|---|---|---|
| bin-1 | RAG-693 | 8.12 | 48.20 |
| | RAG-834 | 8.07 | 9.64 |
| | RAG-692 | 7.69 | 29.53 |
| | RAG-845 | 6.67 | 7.15 |
| | RAG-688 | 6.55 | 42.91 |
| bin-2 | RAG-531 | 2.06 | 3.11 |
| | RAG-705 | 2.05 | 11.33 |
| | RAG-322 | 2.04 | 7.53 |
| | RAG-840 | 2.03 | 7.01 |
| | RAG-741 | 2.02 | 5.45 |
| bin-3 | RAG-883 | 1.00 | 1.76 |
| | RAG-177 | 1.00 | 0.31 |
| | RAG-530 | 1.00 | 1.41 |
| | RAG-879 | 1.00 | 0.98 |
| | RAG-527 | 0.99 | 1.06 |
| bin-4 | RAG-830 | 0.72 | 0.45 |
| | RAG-419 | 0.71 | 0.41 |
| | RAG-420 | 0.71 | 0.93 |
| | RAG-700 | 0.69 | 0.32 |
| | RAG-589 | 0.66 | 0.80 |

Taken together, the above data indicates that saRNAs can be designed to target selected regions in the p21 promoter to induce p21 expression with certain regions being more sensitive and containing higher percentages of functional saRNA targets.

### Example 2: saRNAs Induce p21 mRNA Expression and Inhibit Cancer Cell Proliferation

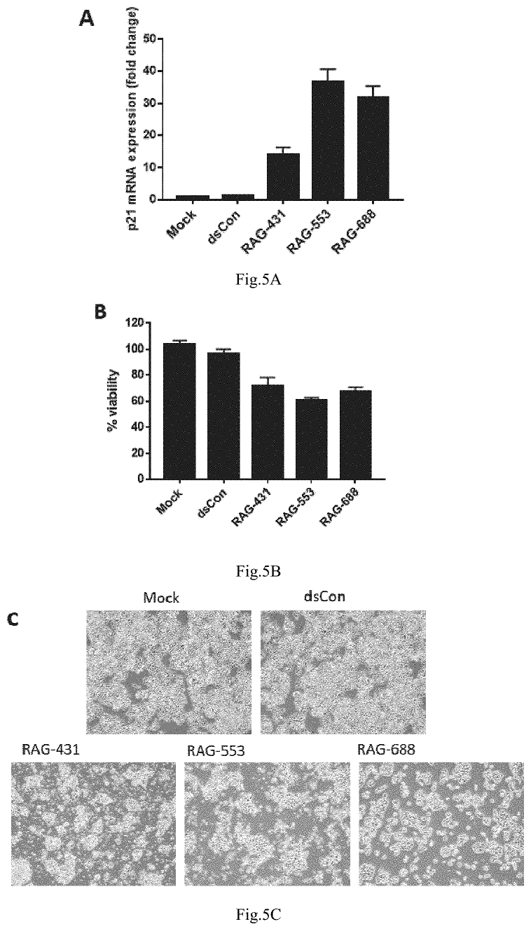

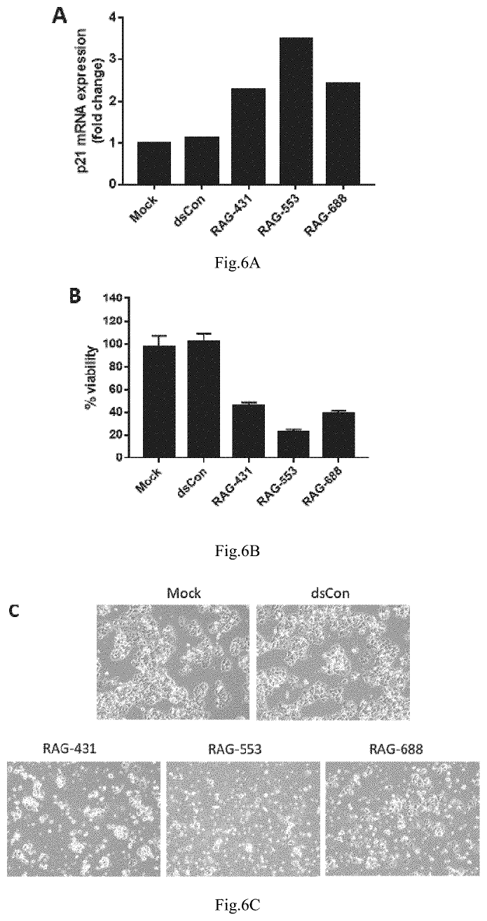

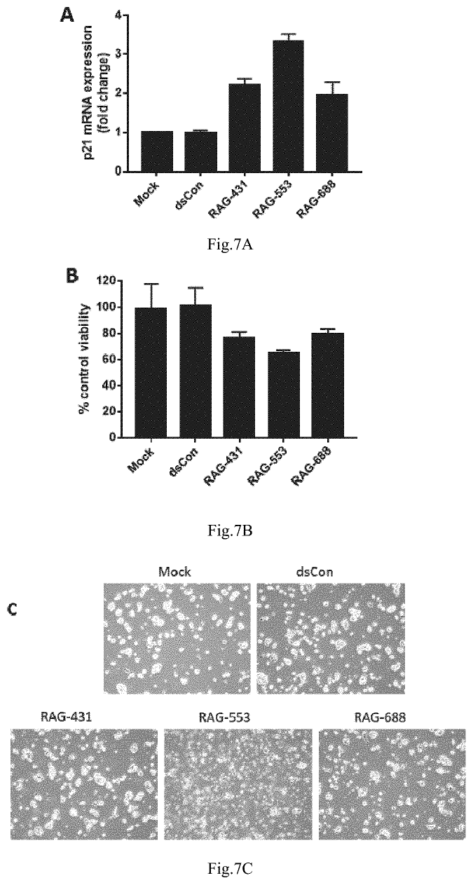

In order to further evaluate the effect of p21 saRNAs in inducing p21 mRNA expression and suppressing cancer cell proliferation, the saRNAs (RAG1-431, RAG1-553, and RAG1-688) screened by QuantiGene 2.0 were transfected into cancer cell lines including KU-7 (bladder cancer), HCT116 (colon cancer), and HepG2 (hepatocellular carcinoma). The result showed that in all the aforementioned cell lines, all saRNAs can induce at least a two-fold change in the p21 mRNA expression levels and suppress cell proliferation, indicating functional activation of p21 protein. Specifically, RAG1-431, RAG1-553, and RAG1-688 were individually transfected into KU-7 cells, caused a 14.0-, 36.9- and 31.9-fold change in the mRNA expression of p21, and exhibited a 71.7%, 60.7% and 67.4% cell survival rate respectively relative to blank control (Mock) (FIG. 5). RAG1-431, RAG1-553, and RAG1-688 were transfected into the HCT116 cells, resulted in a 2.3-, 3.5-, and 2.4-fold change in the mRNA expression of p21, and exhibited a survival rate of 45.3%, 22.5% and 38.5% respectively relative to the blank control (Mock) (FIG. 6). RAG1-431, RAG1-553, and RAG1-688 were transfected into the HepG2 cells, resulted in a 2.2-, 3.3- and 2.0-fold change in the mRNA expression of p21, and exhibited a survival rate of 76.7%, 64.9%, and 79.9% relative to the blank control (Mock) (FIG. 7).

### INCORPORATION BY REFERENCE

All disclosures of each patent literature and scientific literature cited herein are incorporated herein by reference for all purposes.

### EQUIVALENCE

The present invention can be implemented in other specific forms without departing from its fundamental characteristics. Therefore, the aforementioned examples shall be considered as illustrative rather than restrictive to the present invention described herein. The scope of the present invention is represented by the appended claims rather than the above specification, and is intended to cover all changes falling into the meanings and scopes of equivalents of the claims.

1. A saRNA, wherein one strand of the saRNA has at least 75% homology or complementarity with any continuous fragment of 16 to 35 nucleotides in length in a nucleic acid sequence selected from the group consisting of SEQ ID NOs: 5, 6, 7, 8, 9, 10, 11, and 12, and wherein the saRNA activates or upregulates the expression of p21 by targeting a sequence of a human p21 promoter.

2. The saRNA of claim 1, wherein the saRNA comprises a sense nucleic acid strand and an antisense nucleic acid strand, the sense nucleic acid strand and the antisense nucleic acid strand contain complementary regions capable of forming a double-stranded nucleic acid structure, and the sense nucleic acid strand or the antisense nucleic acid strand has more than 75%, more than 80%, more than 90%, more than 95%, more than 99%, or 100% homology with any continuous fragment of 16 to 35 nucleotides in length in a sequence of a human p21 promoter.

3. The saRNA of claim 2, wherein the sense nucleic acid strand and the antisense nucleic acid strand are on two different nucleic acid strands.

4. The saRNA of claim 2, wherein the sense nucleic acid strand and the antisense nucleic acid strand are on the same nucleic acid strand, forming a hairpin single-stranded nucleic acid molecule, wherein the complementary regions of the sense nucleic acid strand and the antisense nucleic acid strand form a double-stranded nucleic acid structure.

5. The saRNA of claim 3, wherein at least one strand of the saRNA has a 3′ overhang of 0 to 6 nucleotides in length.

6. The saRNA of claim 5, wherein both strands of the saRNA have a 3′ overhang of 2 to 3 nucleotides in length.

7. The saRNA of claim 2, wherein the sense nucleic acid strand or the antisense nucleic acid strand is 16 to 35 nucleotides in length.

8. The saRNA of claim 1, wherein the sense nucleic acid strand or the antisense nucleic acid strand has at least 75% homology with a nucleic acid sequence selected from the group consisting of SEQ ID NOs: 13-30, 35-46, 59-62, 67-74, 77-80, 85-96, 103-108, 111-118, 121-132, 139-140, 147-180, 185-186, 189-190, 195-198, 201-202, 209-212, 215-218, 225-240, 243-246, 249-258, 261-262, 265-270, 275-280, 283-300, 303-308, 317-320, 323-324, 329-348, 351-352, 357-358, 361-366, 371-392, 399-400, 405-412, 415-416, 419-424, 429-432, 439-442, 447-450, 453-458, and 463-468.

9. The saRNA of claim 8, wherein the nucleotide sequence of the sense nucleic acid strand or the antisense nucleic acid strand is selected from the group consisting of SEQ ID NOs: 13-30, 35-46, 59-62, 67-74, 77-80, 85-96, 103-108, 111-118, 121-132, 139-140, 147-180, 185-186, 189-190, 195-198, 201-202, 209-212, 215-218, 225-240, 243-246, 249-258, 261-262, 265-270, 275-280, 283-300, 303-308, 317-320, 323-324, 329-348, 351-352, 357-358, 361-366, 371-392, 399-400, 405-412, 415-416, 419-424, 429-432, 439-442, 447-450, 453-458, and 463-468.

10. The saRNA of claim 1, wherein at least one nucleotide of the saRNA is a chemically modified nucleotide, and the chemical modification is at least one of the following modifications:
(1) modification of a phosphodiester bond connecting nucleotides in the nucleotide sequence of the saRNA;
(2) modification of 2′-OH of ribose in the nucleotide sequence of the saRNA;
(3) modification of a base in the nucleotide sequence of the saRNA; or
(4) at least one nucleotide in the nucleotide sequence of the small activating nucleic acid molecule being a locked nucleic acid.

11. The saRNA of claim 1, wherein the expression of p21 is activated or upregulated by at least 10%.

12. A composition comprising the saRNA of claim 1 and a pharmaceutically acceptable carrier.

13-16. (canceled)

17. An isolated p21 saRNA target site, wherein the target site is any continuous 16-35 nucleotide sequence selected from the group consisting of SEQ ID NOs. 5-12.

18. A method for activating or upregulating the expression of p21 in a cell, wherein the method comprises administrating the composition of claim 12.

19. The method of claim 18, wherein the composition is introduced into the cell directly.

20. The method of claim 18, wherein the cell is a mammalian cell.

21. (canceled)

22. The composition of claim 12, wherein the pharmaceutically acceptable carrier is a liposome, a macromolecular polymer, or a polypeptide.

23. A use of the composition of claim 21 in the preparation of a formulation for activating or upregulating p21 expression for treating a tumor or a benign proliferative lesion.

24. The method of claim 20, wherein the mammalian cell is present in a human subject, wherein the subject comprises a cancer caused by insufficient p21 protein expression, and wherein introduction of an effective amount of the composition can treat the cancer.

25. The method of claim 24, wherein the cancer is selected from a bladder cancer, a prostate cancer, a hepatocellular carcinoma, or a colorectal cancer.
